(19) United States
(12) Patent Application Publication
(10) Pub. No.: US 2024/0117426 A1
(43) Pub. Date: Apr. 11, 2024

(54) NANOGEL PARTICLES HAVING DUAL FUNCTIONALITY AND TEMPERATURE RESPONSIVENESS FOR PARTICLE CLUSTERING IN NUCLEIC ACID SEQUENCING SYSTEMS

(71) Applicants: Illumina, Inc., San Diego, CA (US); Illumina Cambridge Limited, Cambridge (GB)

(72) Inventors: Nam Nguyen, Cambridge (GB); Xavier von Hatten, Cambridge (GB); Will Tovey, Bury St Edmunds (GB); Andrew Brown, Cambridge (GB); Wayne George, Haverhill (GB); Eric Brustad, San Diego, CA (US); Gianluca Artioli, Cambridge (GB)

(73) Assignees: Illumina, Inc., San Diego, CA (US); Illumina Cambridge Limited, Cambridge (GB)

(21) Appl. No.: 18/469,440

(22) Filed: Sep. 18, 2023

Related U.S. Application Data
(60) Provisional application No. 63/407,852, filed on Sep. 19, 2022.

(51) Int. Cl. C12Q 1/6874; C08F 120/06; C08F 120/56; C08F 120/60; C08F 122/38; C08F 138/02

(52) U.S. Cl. CPC C12Q 1/6874; C08F 120/06; C08F 120/56; C08F 120/60; C08F 122/385; C08F 138/02

(57) ABSTRACT

In some examples, novel nanogel particles are described having dual functionality, temperature responsiveness and pH responsiveness. For nucleic acid sequencing, amplification primers are grafted to nanogel particles to form primer-grafted nanogel particles, and the primer-grafted nanogel particles are captured onto surfaces within a flow cell. Within flow cells such as used in SBS nucleic acid sequencing, each primer-grafted nanogel particle functions as a nano-well in the flow cell, thus eliminating the need for nano-wells in some examples.

### CROSS-REFERENCE TO RELATED APPLICATIONS

This application claims the benefit of U.S. Provisional Patent Application No. 63/407,852, filed Sep. 19, 2022 and entitled “NANOGEL PARTICLES HAVING DUAL FUNCTIONALITY AND TEMPERATURE RESPONSIVENESS FOR PARTICLE CLUSTERING IN NUCLEIC ACID SEQUENCING SYSTEMS,” the entire contents of which are incorporated by reference herein.

### FIELD

The present disclosure generally relates to nucleic acid sequencing methods and devices and in particular to functionalized nanogel particles usable in sequencing by synthesis (SBS) methods.

### BACKGROUND

Nucleic acid sequencing continues to be an important tool in a multitude of diverse fields, from ancestry to law enforcement to medical diagnostics. Sequencing methods today are rapid and cost effective. Nonetheless, there is a continuing need to drive genetic sequencing costs down even further, such as by streamlining individual process steps in sequencing methods and improving various devices, such as flow cells used in Sequencing by Synthesis (SBS).

### SUMMARY

Nanogel particles having dual functionality and temperature and/or pH responsiveness for particle clustering in nucleic acid sequencing systems are provided herein.

For example, as provided herein, certain polymeric nanogel particles may be used to replace hydrogel coatings in flow cells for Sequencing by Synthesis (SBS). Nanogel particles also or alternatively may be used to improve many aspects of an SBS method.

In various examples provided herein, a nanogel particle functions as a replacement for a nano-well in a flow cell, thus eliminating the need to configure nano-wells in the flow cells for SBS. Other examples may include an operation trapping nanogel particles in nano-wells configured in a flow cell.

In various examples provided herein, sequencing on a nanogel particle versus a hydrogel surface improves the monoclonality of the clustering of multiple copies of a sequencing template. For example, confining the clustering to a nano scale particle may improve signal to noise ratio, error rate, and overall quality and coverage of genome during sequencing.

In various examples, nanogel particles disclosed herein exhibit dual functionality through the presence of at least two types of reactive end groups on copolymer chains within the nanogel particles. In various examples, nanogel particles disclosed herein exhibit temperature responsiveness wherein the nanogel particles can shrink or swell in response to temperature changes, and pH responsiveness wherein the nanogel particles may include at least some copolymer chains with carboxylic acid end groups that are more nonionic in character in certain pH ranges and more anionic in character in other pH ranges.

Dual functionality and dual responsiveness (temperature/pH) characteristics of nanogel particles, such as provided herein, allows initial attachment of alkyne-functionalized amplification primers onto the particles using, for example, —N₃ functionality on the particles, while pH responsiveness enhances chemical capture onto flow cell surfaces using bioconjugation techniques. These amplification primer-functionalized nanogel particles are demonstrated to support on-board particle clustering and SBS sequencing.

In various examples of the present disclosure, polymeric nanogel particles are described. Polymeric nanogel particles comprise copolymer chains further comprising a first recurring unit of Formula (I),

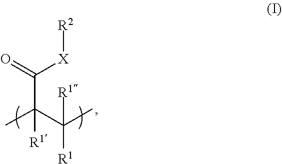

wherein each of R¹, R^{1′}, and R¹″ is independently selected from H, halogen, alkyl, alkoxy, alkenyl, alkynyl, cycloalkyl, aryl, heteroaryl, or heterocyclyl; X is —O— or —NH—; and R² is —CH₂—C≡CH or has a structure:

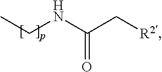

wherein R^{2′} is —N₃ or —C≡CH; and p is an integer of 1 to 50;
and a second recurring unit of Formula (II),

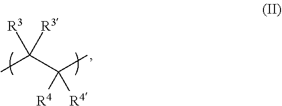

wherein each of R³, R^{3′}, R⁴, and R^{4′} is independently selected from —H, —R⁵, —OR⁵, —CO₂R⁵, —C(O)R⁵, —OC(O)R⁵, —C(O)NR⁶R⁷, and —NR⁶R⁷; wherein R⁵ is —H, —OH, alkyl, cycloalkyl, hydroxyalkyl, aryl, heteroaryl, or heterocyclyl; and each of R⁶ and R⁷ is independently selected from —H and alkyl; wherein at least some of the copolymer chains include at least one carboxylic acid end group; and wherein at least some of the copolymer chains include at least one —N₃ or —C≡CH end group.

In various examples, R¹, R^{1′}, and R^{1″} are each H.

In various examples, R¹ and R^{1″} are H, and R^{1′} is CH₃.

In various examples, the first recurring unit of Formula (I) is:

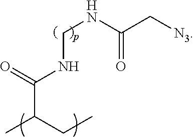

In various examples, Formula (I) is:

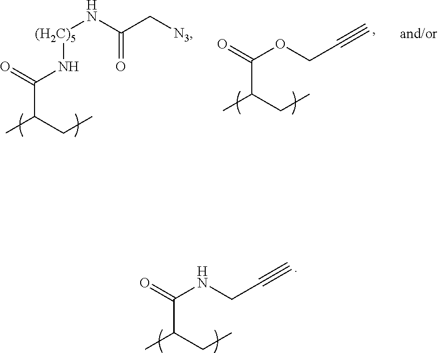

In various examples, the second recurring unit of Formula (II) is at least one of:

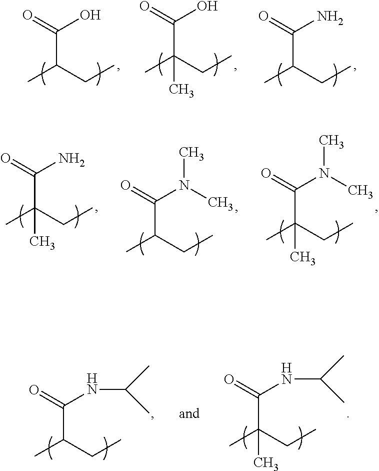

In various examples, polymeric nanogel particles are derived from a monomer mixture comprising N-(5-(2-azidoacetamido)pentyl)acrylamide (AzAPA); N-isopropylacrylamide (NiPAM); acrylic acid (AAc); and N,N′-methylenebisacrylamide (BisAM).

In various examples, nanogel particles comprise poly(AzAPA-co-NiPAM-co-AAc-co-BisAM) copolymer chains.

In various examples, polymeric nanogel particles are derived from a monomer mixture comprising propargyl acrylate (PAG) and/or N-propargyl acrylamide (PAM); N-isopropylacrylamide (NiPAM); acrylic acid (AAc); and N,N′-methylenebisacrylamide (BisAM).

In various examples, polymeric nanogel particles comprise poly(PAG-co-NiPAM-co-AAc-co-BisAM) and/or poly(PAM-co-NiPAM-co-AAc-co-BisAM) copolymer chains.

In various embodiments of the present disclosure, a substrate has a surface comprising a plurality of pH and temperature responsive organic polymeric nanogel particles covalently attached thereto, wherein the organic polymeric nanogel particles include a plurality of copolymer chains comprising copolymer chains further comprising a first recurring unit of Formula (I),

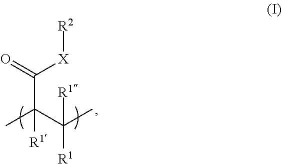

wherein each of R¹, R^{1′}, and R^{1″} is independently selected from H, halogen, alkyl, alkoxy, alkenyl, alkynyl, cycloalkyl, aryl, heteroaryl, or heterocyclyl; X is —O— or —NH—; and R² is —CH₂—C≡CH or has a structure:

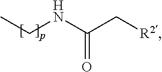

wherein R^{2′} is —N₃ or —C≡CH; and p is an integer of 1 to 50;
and a second recurring unit of Formula (II),

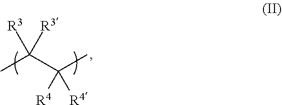

wherein each of R³, R^{3′}, R⁴, and R^{4′} is independently selected from —H, —R⁵, —OR⁵, —CO₂R⁵, —C(O)R⁵, —OC(O)R⁵, —C(O)NR⁶R⁷, and —NR⁶R⁷; wherein R⁵ is —H, —OH, alkyl, cycloalkyl, hydroxyalkyl, aryl, heteroaryl, or heterocyclyl; and each of R⁶ and R⁷ is independently selected from —H and alkyl; wherein at least some of the copolymer chains include at least one carboxylic acid end group; and wherein at least some of the copolymer chains include at least one —N₃ or —C≡CH end group.

In various examples, organic polymer nanogel particles are formed from a monomer mixture comprising (a) propargyl acrylate (PAG) and/or N-propargyl acrylamide (PAM), (b) N-isopropylacrylamide (NiPAM); and (c) acrylic acid (AAc).

In various examples, organic polymer nanogel particles have an average size of about 265 nm to about 280 nm.

In various examples, organic polymer nanogel particles are formed from a monomer mixture comprising N-(5-(2-azidoacetamido)pentyl)acrylamide (AzAPA), N-isopropylacrylamide (NiPAM), and acrylic acid (AAc).

In various examples, organic polymer nanogel particles have an average size of about 225 nm to about 250 nm.

In various examples, a monomer mixture used in the synthesis of organic polymer nanogel particles further comprises a Multi-functional compound selected from the group consisting of N,N′-methylenebisacrylamide, N,N′-mnethylenebismethacrylanide, polyethylene glycol diacrylate, polyethylene glycol dimethacrylate, N-vinylacrylamide, glycidyl acrylate, divinylbenzene, tetraallyl ammonium chloride, diallyl dimethyl ammonium chloride, and mixtures thereof.

In various examples, the covalent attachment between a substrate and a plurality of nanogel particles comprise amide —NH—C(O)— linkages, wherein the —NH— portion of each amide linkage originated as an —NH₂ group present in a plurality of —NH₂ groups on the substrate, and wherein the —C(O)— portion of each amide linkage originated as a carboxylic acid end group on a respective copolymer chain.

In various examples, organic polymeric nanogel particles further comprise amplification primers grafted thereon.

In various examples, each graft of an amplification primer to an organic polymeric nanogel particle comprises a triazine linkage, formed from a click-chemistry reaction between a terminal alkyne substituent on the amplification primer and an azide group on an end of a respective copolymer chain, or a click-chemistry reaction between a terminal azide substituent on the amplification primer and an alkyne group on an end of a respective copolymer chain.

In various examples, the first recurring unit of Formula (I) is:

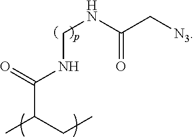

In various examples, the first recurring unit of Formula (I) is:

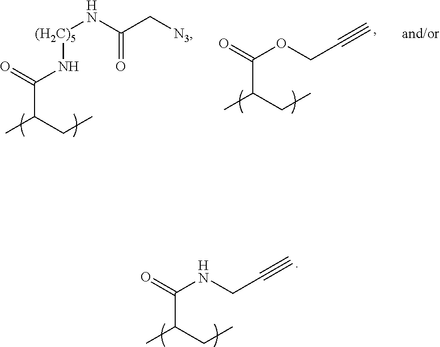

In various examples, the second recurring unit of Formula (II) is at least one of:

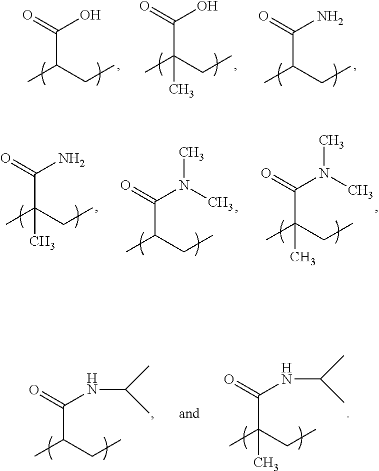

In various embodiments of the present disclosure, a flow cell is described that can be used in nucleic acid sequencing. In various examples, a flow cell comprises a substrate having a surface comprising a plurality of pH and temperature responsive organic polymeric nanogel particles covalently attached thereto, wherein the organic polymeric nanogel particles include a plurality of copolymer chains having both a first recurring unit of Formula (I) and a second recurring unit of Formula (II), and wherein the organic polymeric nanogel particles further comprise amplification primers grafted thereon.

In various examples, a method of synthesizing organic polymeric nanogel particles comprises reacting an aqueous dispersion of N-(5-(2-azidoacetamido)pentyl)acrylamide (AzAPA), N-isopropylacrylamide (NiPAM), acrylic acid (AAc), and N,N′-methylenebisacrylamide (BisAM) monomers under conditions suitable for suspension/precipitation free-radical polymerization in the presence of a dispersant and a free-radical initiator, wherein the polymeric nanogel particles thus synthesized comprise poly(AzAPA-co-NiPAM-co-AAc-co-BisAM) copolymer chains.

In various examples, a method of synthesizing organic polymeric nanogel particles comprises reacting an aqueous dispersion of propargyl acrylate (PAG), N-isopropylacrylamide (NiPAM), acrylic acid (AAc), and N,N′-methylenebisacrylamide (BisAM) monomers under conditions suitable for suspension/precipitation free-radical polymerization in the presence of a dispersant and a free-radical initiator, wherein the polymeric nanogel particles thus synthesized comprise poly(PAG-co-NiPAM-co-AAc-co-BisAM) copolymer chains.

In various examples, a method of synthesizing organic polymeric nanogel particles comprises reacting an aqueous dispersion of propargyl acrylamide (PAM), N-isopropylacrylamide (NiPAM), acrylic acid (AAc), and N,N′-methylenebisacrylamide (BisAM) monomers under conditions suitable for suspension/precipitation free-radical polymerization in the presence of a dispersant and a free-radical initiator, wherein the polymeric nanogel particles thus synthesized comprise poly(PAM-co-NiPAM-co-AAc-co-BisAM) copolymer chains

In various examples, the dispersant comprises sodium dodecyl sulfate.

In various examples, the free-radical initiator comprises ammonium persulfate.

In various examples, a method for assembling a flow cell usable in sequencing nucleic acids, the method comprises:

(a) preparing a plurality of organic polymeric nanogel particles, each nanogel particle including copolymer chains comprising a recurring unit of Formula (I) and a recurring unit of Formula (II),

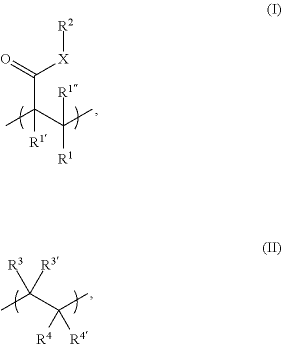

wherein each of R¹, R^{1′}, and R^{1″} is independently selected from H, halogen, alkyl, alkoxy, alkenyl, alkynyl, cycloalkyl, aryl, heteroaryl, or heterocyclyl; X is —O— or —NH—; R² is —CH₂—C≡CH or having a structure:

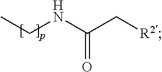

wherein each of R³, R^{3′}, R⁴, and R^{4′} is independently selected from —H, —R⁵, —OR⁵, —CO₂R⁵, —C(O)R⁵, —OC(O)R⁵, —C(O)NR⁶R⁷, and —NR⁶R⁷; R⁵ is —H, —OH, alkyl, cycloalkyl, hydroxyalkyl, aryl, heteroaryl, or heterocyclyl; wherein each of R⁶ and R⁷ is independently selected from —H and alkyl; R^{2′} is —N₃ or —C≡CH; and p is an integer of 1 to 50; wherein at least some of the copolymer chains include at least one carboxylic acid end group; and wherein at least some of the copolymer chains include at least one —N₃ or —C≡CH end group;
(b) grafting amplification primers onto the organic polymeric nanogel particles by performing click-chemistry reactions between a —C≡CH group on an alkyne-functionalized amplification primer and an —N₃ end group on a respective copolymer chain, or performing click-chemistry reactions between a —N₃ group on an azide-functionalized amplification primer and an —C≡CH end group on a respective copolymer chain; and
(c) binding the primer-grafted organic polymeric nanogel particles onto designated regions of a surface of the flow cell through amide —NH—C(O)— linkages by conducting temperature and pH controlled amide condensation reactions, wherein the —NH— portion of each amide linkage originated as an —NH₂ group present in a plurality of —NH₂ groups on the designated regions of the surface, and wherein the —C(O)— portion of each amide linkage originated as a carboxylic acid end group on a respective copolymer chain.

In various examples, preparing the organic polymeric nanogel particles comprises reacting an aqueous dispersion of N-(5-(2-azidoacetamido)pentyl)acrylamide (AzAPA), N-isopropylacrylamide (NiPAM), acrylic acid (AAc), and N,N′-methylenebisacrylamide (BisAM) monomers under conditions suitable for suspension/precipitation free-radical polymerization in the presence of a dispersant and a free-radical initiator, wherein the polymeric nanogel particles thus synthesized comprise poly(AzAPA-co-NiPAM-co-AAc-co-BisAM) copolymer chains.

In various examples, preparing the organic polymeric nanogel particles comprises reacting an aqueous dispersion of propargyl acrylate (PAG), N-isopropylacrylamide (NiPAM), acrylic acid (AAc), and N,N′-methylenebisacrylamide (BisAM) monomers under conditions suitable for suspension/precipitation free-radical polymerization in the presence of a dispersant and a free-radical initiator, wherein the polymeric nanogel particles thus synthesized comprise poly(PAG-co-NiPAM-co-AAc-co-BisAM) copolymer chains.

In various examples, preparing the organic polymeric nanogel particles comprises reacting an aqueous dispersion of propargyl acrylamide (PAM), N-isopropylacrylamide (NiPAM), acrylic acid (AAc), and N,N′-methylenebisacrylamide (BisAM) monomers under conditions suitable for suspension/precipitation free-radical polymerization in the presence of a dispersant and a free-radical initiator, wherein the polymeric nanogel particles thus synthesized comprise poly(PAM-co-NiPAM-co-AAc-co-BisAM) copolymer chains.

In various examples, grafting of amplification primers onto the organic polymeric nanogel particles in (b) above further includes swelling of the organic polymeric nanogel particles prior to said grafting by reducing a temperature of the amide condensation reactions to less than about 20° C.

In various examples, binding of the grafted organic polymeric nanogel particles onto the designated regions of the surface of the flow cell in (c) above is preceded by physically capturing the grafted organic polymeric nanogel particles from a solution of grafted organic polymeric nanogel particles onto the designated regions of the surface of the flow cell by performing the steps of (i) shrinking the grafted organic polymeric nanogel particles by increasing a temperature of the solution to about 60° C.; (ii) locating the shrunken grafted organic polymeric nanogel particles into the designated regions of the surface of the flow cell; and (iii) swelling the located grafted organic polymeric nanogel particles by decreasing the temperature of the solution to less than about 20° C. to physically trap the grafted organic polymeric nanogel particles onto the designated regions of the surface of the flow cell.

In various examples, designated regions of the surface of the flow cell comprise nano-wells patterned in a substrate.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF THE DRAWINGS

FIG. 1A schematically illustrates an example procedure for preparing ANA nanogel particles including poly(AzAPA-co-NiPAM-co-AAc-co-BisAM) copolymer chains, synthesized under suspension/precipitation polymerization conditions in accordance with various examples of the present disclosure.

FIG. 1B illustrates example temperature dependent shrinking/swelling characteristics of example nanogel particles including poly(AzAPA-co-NiPAM-co-AAc-co-BisAM) copolymer chains.

FIG. 1C schematically illustrates grafting of alkyne-P5/P7 amplification primers onto example nanogel particles including poly(AzAPA-co-NiPAM-co-AAc-co-BisAM) copolymer chains under CuAAc conditions to form ANA-P5P7 primer-grafted nanogel particles, and a plot showing the temperature dependent size of the example ANA-P5P7 grafted nanogel particles.

FIG. 2A schematically illustrates capture of ANA-P5P7 primer-grafted nanogel particles onto a CMS-treated PAZAM coating within a SBS flow cell in the presence of DMTMM activation.

FIGS. 2B-2D illustrate example SBS sequencing and SBS metrics thus obtained using ANA-P5P7 primer-grafted nanogel particles in an SBS flow cell.

FIG. 3A schematically illustrates an example procedure for preparing PANA nanogel particles including poly(PAG-co-NiPAM-co-AAc-co-BisAM) copolymer chains, synthesized under suspension/precipitation polymerization conditions in accordance with various examples of the present disclosure.

FIG. 3B illustrates example temperature dependent shrinking/swelling characteristics of example nanogel particles including poly(PAG-co-NiPAM-co-AAc-co-BisAM) copolymer chains.

FIG. 3C schematically illustrates various example reactions from PANA nanogel particles including poly(PAG-co-NiPAM-co-AAc-co-BisAM) copolymer chains, including grafting of amplification primers on the nanogel particles and capture of nanogel particles onto untreated and treated flow cell surfaces.

FIG. 4A schematically illustrates click-chemistry reactions of FITC dye to —C≡CH end groups present on PANA nanogel particles having —C≡CH end groups, using N₃-FITC with CuAAC chemistry or UV-activated click-chemistry reaction between HS-PEG₁₂ₖ-FITC and PANA particles

FIG. 4B illustrates example PANA nanogel particle capture into nano-wells of a flow cell via CuAAC chemistry or DMTMM bioconjugation to CMS treated PAZAM.

FIG. 5A schematically illustrates grafting of N₃-P5/P7 amplification primers onto nanogel particles including poly(PAG-co-NiPAM-co-AAc-co-BisAM) copolymer chains under CuAAc conditions to form PANA-P5P7 primer-grafted nanogel particles.

FIG. 5B illustrates two alternative example methods for capturing PANA-P5P7 particles onto flow cell surfaces utilizing either of two different chemistries. In this scenario, amplification primers can be grafted onto the nanogel particles after capture on surfaces.

FIG. 5C shows the C1 intensity heat map of the flow cell of FIG. 5A compared to the CFR QC fluorescence intensity shown in FIG. 5B.

FIG. 5D sets forth example SBS intensities and metrics obtained for primer-grafted PANA-P5P7 particles when used in an SBS flow cell.

FIG. 6A illustrates additional examples using nanogel particles in accordance with the present disclosure, including forming peptide-functionalized PANA particles and nanoparticle-protein corona therefrom.

FIG. 6B illustrates an example procedure for preparing N-propargyl acrylamide (PAM) in accordance with various examples of the present disclosure.

### DETAILED DESCRIPTION

The detailed description of examples herein makes reference to the accompanying drawings, which show examples by way of illustration and their best mode. While these examples are described in sufficient detail to enable those skilled in the art to practice the present subject matter, it should be understood that other examples may be realized and that logical, chemical, and mechanical changes may be made without departing from the spirit and scope of the subject matter provided herein. Thus, the detailed description is presented for purposes of illustration only and not of limitation. For example, unless otherwise noted, the steps recited in any of the method or process descriptions may be executed in any order and are not necessarily limited to the order presented. Furthermore, any reference to singular element or step includes plural element or step, and any reference to more than one component or step may include a singular element or step. Also, any reference to attached, fixed, connected or the like may include permanent, removable, temporary, partial, full and/or any other possible attachment option. Additionally, any reference to without contact (or similar phrases) may also include reduced contact or minimal contact.

### Terms

As used herein, the term “nanogel particle” is intended to refer to a nanoscale polymeric particle including copolymer chains that are optionally crosslinked. Simply for the sake of convenience, nanogel particles herein may be illustrated as “soccer balls,” i.e., substantially spherical in shape, although their structure might not be this simple. A spherical representation allows the reader to understand the concept of accessible functional groups in/on a nanogel particle since these groups, typically functional end groups on copolymer chains, can be shown to protrude from the surface of the particle. Nonetheless, particle size analysis can be performed, such as by light scattering, to obtain relevant particle size distributions or Z-averages. So even though nanogel particles herein may not be entirely spherical in shape with functional groups protruding from a surface, their average size can be determined. Typically, nanogel particles in accordance with the present disclosure have Z-averages of about 50 nm to about 500 nm. Also, recitations herein describe chemical reactions as taking place on a nanogel particle, at least for simplicity. Although nanogel particles herein are likely spherical in shape due to the method of synthesis comprising suspension/precipitation polymerization, the present disclosure is not limited in regard to particle shape. All nanogel “objects,” regardless of shape, are within the scope of the present disclosure. Further, it should be understood that since the particles comprise lightly crosslinked networks containing mostly water, various chemical reactions may take place both on and in a nanogel particle.

As used herein, the term “dual functionality” is intended to refer to a property or characteristic of nanogel particles when the nanogel particles include at least some copolymer chains having two types of functional substituents present as copolymer chain end groups, such as carboxylic acid end groups, and —N₃ or —C≡CH end groups. Each type of functional group can be used in specific binding or conjugation reactions. For example, nanogel particles having dual functionality allow covalent attachment of amplification primers onto the nanogel particles (such as by reacting alkyne-functionalized amplification primers with the free —N₃ end groups present on the copolymer chains of the nanogel particles) and binding of dual functionalized nanogel particles to surfaces in a flow cell (such as by reacting the free carboxylic acid end groups on the copolymer chains of the nanogel particles with functionalized groups appended to the flow cell surfaces).

As used herein, the term “temperature responsiveness” is intended to refer to a property or characteristic of nanogel particles when the nanogel particles include at least some copolymer chains having sections of polymer structure physically responsive to temperature. More specifically, nanogel particles that are temperature responsive exhibit shrinking when exposed to increasing or decreasing temperatures, and exhibit swelling when exposed to the opposite temperature trend. In various examples, nanogel particles having copolymer chains with blocks of poly(NiPAM) shrink with increasing temperature. This temperature responsiveness provides methods for placing nanogel particles into holes, such as nano-wells, and then locking them in place simply by temperature manipulations.

As used herein, the term “pH responsiveness” is intended to refer to a property or characteristic of nanogel particles when the nanogel particles include at least some copolymer chains having carboxylic acid end groups, such that at certain pH ranges these groups are predominantly —CO₂H, and such that at other pH ranges these groups are predominantly —CO₂. Stated another way, pH responsive carboxylic acid end groups on at least some of the copolymer chains of the nanogel particles impart pH responsiveness to the nanogel particles. In various examples, the pH responsiveness allows for pH-driven binding of nanogel particles to functionalized flow cell surfaces.

As used herein, the term “dual stimuli (temperature/pH)” is intended to refer to the combination of temperature responsiveness and pH responsiveness properties (per the above definitions) exhibited by certain nanogel particles. In various examples, blocks of poly-NiPAM in copolymer chains of a nanogel particle imparts temperature responsiveness to the nanogel particles (i.e., shrinking/swelling), whereas the presence of AAc units in copolymer chains of a nanogel particle contributes to the pH responsiveness of the nanogel particles.

As used herein, the term “suspension/precipitation polymerization” is intended to refer to a free-radical suspension polymerization reaction in which water-soluble monomers and a free-radical initiator produce the polymeric nanogel particles as a dispersed solid phase when using a dispersant or steric stabilizer and vigorous stirring of the reaction mixture. Suspension/precipitation polymerization is thoroughly explained in the academic reference, S. Beck, et al., Chapter 3, pp 21-85 in “Polymer Science and Nanotechnology-Fundamentals and Applications,” Elsevier, 2020, https://doi.org/10.1016/B978-0-12-816806-6.00003-0, the entire contents of which are incorporated by reference herein. Further, the present disclosure is not limited to this particular polymerization method for the synthesis of nanogel particles. For example, emulsion polymerization techniques may be employed, and non-aqueous solvents may be used.

As used herein, the acronym “AzAPA” is intended to refer to the monomer, N-(5-(2-azidoacetamido)pentyl)acrylamide.

As used herein, the acronym “NiPAM” is intended to refer to the monomer, N-isopropylacrylamide.

As used herein, the acronym “BisAM” is intended to refer to the multifunctional monomer, N,N′-methylenebisacrylamide.

As used herein, the acronym “PAG” is intended to refer to the monomer, propargyl acrylate.

As used herein, the acronym “PAM” is intended to refer to the monomer, N-propargyl acrylamide.

As used herein, the acronym “AAc” is intended to refer to the monomer, acrylic acid.

As used herein, the acronym “BrAPA” is intended to refer to the monomer N-(5-(2-bromoacedamido)pentyl)acrylamide, used in various examples to form PAZAM coatings on flow cell (FC) surfaces.

As used herein, the acronym “SDS” is intended to refer to the anionic dispersant sodium dodecyl sulfate.

As used herein, the acronym “APS” is intended to refer to the free-radical polymerization initiator ammonium persulfate.

As used herein, the acronym “ANA” is intended to refer to nanogel particles including poly(AzAPA-co-NiPAM-co-AAc-co-BisAM) copolymer chains. ANA particles feature both carboxylic acid and —N₃ end groups on at least some copolymer chains.

As used herein, the acronym “PANA” is intended to refer to nanogel particles including poly(PAG-co-NiPAM-co-AAc-co-BisAM) copolymer chains. PANA particles feature both carboxylic acid and —C≡CH end groups on at least some copolymer chains.

As used herein, the acronym “PANA”′ is intended to refer to nanogel particles including poly(PAM-co-NiPAM-co-AAc-co-BisAM) copolymer chains. PANA' particles feature both carboxylic acid and —C≡CH end groups on at least some copolymer chains.

As used herein, the term “flow cell” (and acronym “FC”) is intended to refer to a vessel having a chamber (e.g., a flow channel or “lane”) where a reaction can be carried out, an inlet for delivering reagent(s) to the chamber, and an outlet for removing reagent(s) from the chamber. In various examples, the chamber enables the detection of the reaction that occurred in the chamber. For example, the chamber can include one or more transparent surfaces allowing for the optical detection of arrays, optically labeled molecules, or the like, in the chamber. In various examples, polymeric materials such as nanogel particles or hydrogel polymer coatings may be attached to surfaces in a flow cell channel.

As used herein, the term “covalently attached” or “covalently bonded” is intended to refer to the forming of a chemical bonding that is characterized by the sharing of pairs of electrons between atoms. For example, a covalently attached polymer coating is intended to refer to a polymer coating that forms chemical bonds with a functionalized surface of a substrate, as compared to attachment to the surface via other means, for example, adhesion or electrostatic interaction. It will be appreciated that polymers that are attached covalently to a surface can also be bonded via means in addition to covalent attachment.

As used herein, the acronym “PAZAM” is intended to refer to a functionalized polymeric coating including poly(N-(5-azidoacetamidylpentyl) acrylamide-co-acrylamide.

As used herein, the acronym “DMTMM” is intended to refer to the compound, 4-(4,6-Dimethoxy-1,3,5-triazin-2-yl)-4-methylmorpholinium chloride.

As used herein, the acronym “CuAAC” is intended to refer to copper-catalyzed azide-alkyne cycloaddition click-chemistry.

As used herein, the acronym “dz” or “Dz” (such as may be found in various drawing figures herein) is intended to refer to a “Z-average” reported from a particle size analysis and is known in the art as a reliable measure of the average size of a particle size distribution. A Z-average can be directly ascertained from a light-scattering experiment using a nanoparticle analyzer. See, for example, J. C. Thomas, “The determination of log normal particle size distributions by dynamic light scattering,” J. Colloid Interface Sci., 117(1), 187-192 (1987).

As used herein, the acronym “SBS” is intended to refer to “Sequencing by Synthesis,” a sequencing technology using fluorescently labeled nucleotides to sequence multitudes of clusters present on a flow cell surface in parallel. In some examples of SBS, during each sequencing cycle, a single labeled dNTP is added to the nucleic acid chain. The nucleotide label serves as a terminator for polymerization, such that after each dNTP incorporation, the fluorescent dye is imaged to identify the base and then enzymatically cleaved to allow incorporation of the next nucleotide. Further understanding of SBS is disclosed in PCT Application Publications WO2018/119101 and WO2020/005501 (both to Illumina, Inc.), the disclosures of which are incorporated herein by reference in their entireties.

As used herein, the term “seeding” is intended to refer to binding of a single stranded oligonucleotide (ssDNA) to an amplification primer covalently attached to a nanogel particle. In various examples, seeding includes monoclonal seeding.

As used herein, the term “particle clustering” is intended to refer to clustering of multiple copies of one type (monoclonal) or multiple types (polyclonal) of a sequencing template or templates, respectively, on a single nanogel particle previously grafted with amplification primers and having seeded ssDNA. The term particle clustering is intended to refer to activity on a nanogel particle and is not to be confused with physical clustering of nanogel particles themselves.

As used herein, the term “suspension clustering” is intended to refer to a process whereby nanogel particles, previously seeded with a ssDNA and clustered, are subsequently captured on a FC for sequencing.

As used herein, the term “on-board clustering” is intended to refer to a process whereby nanogel particles of suitable size (e.g., ranging from about 200 nm to about 400 nm), previously grafted with primer density compatible with sequencing and subsequently captured in nano-wells of a FC (e.g., HiSeqX™ platform), are then clustered to generate enough copy of a template usable for sequencing.

As used herein, the term “Typhoon” is intended to refer to the Amersham™ Typhoon™ a commercially available laser-scanner platform from Cytiva Life Sciences, for imaging and quantitation of nucleic acids and proteins. When used as an action verb, the term is intended to refer to performing a method of imaging, such as fluorescent imaging, using the Amersham™ Typhoon™ laser-scanner.

For additional acronyms and terminology relating to hydrogel coatings in flow cells and the use of these flow cells in SBS, see U.S. Pat. No. 10,919,033 (Illumina, Inc.), the disclosure of which is incorporated herein in its entirety.

As used herein, any “R” group designated on a chemical structure, such as, R¹, R², R³, R⁴, R⁵, R⁶, R⁷, and Rand so forth, represent substituents in organic chemistry that can be attached to the indicated atom to which the “R” group is bonded. An R group may be substituted or unsubstituted. If two “R” groups are described as being “taken together” to form a cyclic structure, the R groups and the atoms to which they are attached can form a cycloalkyl, aryl, heteroaryl, or heterocyclic ring. In some instances, the ring thus formed may create a bicyclic or tricyclic structure. Although a trivial name may be used for a particular substituent, it is to be understood that being a substituent it has to have some valency available for attachment to another atom, (e.g., a “carboxylic acid” substituent is more formally the monovalent substituent —CO₂ or —CO₂H).

As used herein, the term “alkyl” is intended to refer to linear or branched monovalent fully saturated hydrocarbon substituents, optionally substituted with one or more functional groups anywhere on the substituent. Unless otherwise specified, an alkyl group may contain any number of carbon atoms, such as for example, C₁-C₂₄, C₁-C₁₈, C₁-C₁₀, C₁-C₈, C₁-C₆, or C₁-C₄. Examples of alkyl substituents include, but are not limited to, methyl, ethyl, n-propyl, isopropyl, n-butyl, isobutyl, sec-butyl, tert-butyl, n-pentyl, iso-pentyl neo-pentyl, n-hexyl, iso-hexyl, octadecyl, dodecyl, and so forth. An alkyl substituent herein may be substituted, i.e., having one or more substituent groups appended on the alkyl group or incorporated within the alkyl chain. A substitution within an alkyl substituent chain may include an ether, sulfide, or imine linkage, i.e., —O—, —S—, or —N═, for example, or some other intervening heteroatom(s). Examples of substitution on an alkyl substituent include, but are not limited to, —CN, —N₃, —NH₂, —NHR, —N(R)₂, —N(R)₃⁺, —NO₂, —NH—NH₂, —NH—NHR, —NH—NR₂, -halo, —SH, —SR, —S(═O)R, —SO₂R, —OPO₃^{2—}, —PO₃^{2—}, —OH, —OR, —C(═O)R, —OC(═O)R, —CO₂R, —NHC(═O)R, —NRC(═O)R, —C(═O)NHR, —C(═O)NR₂, alkyl, alkenyl, cycloalkyl, heterocyclyl, and aryl, wherein each R above is independently selected from hydrogen —H and an alkyl moiety, including, for example, C₁₋₆ alkyl (e.g., —CH₃, —C₂H₅, -isopropyl, -tert-butyl, etc.), C₁₋₆ alkoxy (e.g., —OCH₃, —OC₂H₅), halogenated C₁₋₆ alkyl (e.g., —CF₃, —CHF₂, —CH₂F), and halogenated C₁₋₆ alkoxy (e.g., —OCF₃, —OC₂F₅).

As used herein, the term “cycloalkyl” includes any 3-, 4-, 5-, 6-, 7-, or 8-membered, saturated or unsaturated, non-aromatic carbocyclic ring, optionally substituted with one or more functional groups at any location on the cyclic substituent. Examples of cycloalkyl groups include, but are not limited to, cyclopropyl, cyclobutyl, cyclopentyl, 1-, 2-, or 5-cyclopentadienyl, cyclohexyl, 1-, 3- or 4-cyclohexenyl, 1-, 2-, or 5-(1,3-cyclohexadienyl), 1- or 3-(1,4-cyclohexadienyl), cycloheptyl, 1-, 3-, 4-, or 5-cycloheptenyl, cyclooctanyl, and so forth. Examples of substitution on an cycloalkyl substituent include, but are not limited to, —CN, —N₃, —NH₂, —NHR, —N(R)₂, —N(R)₃⁺, —NO₂, —NH—NH₂, —NH—NHR, —NH—NR₂, -halo, —SH, —SR, —S(═O)R, —SO₂R, —OPO₃^{2—}, —PO₃^{2—}, —OH, —OR, —C(═O)R, —OC(═O)R, —CO₂R, —NHC(═O)R, —NRC(═O)R, —C(═O)NHR, —C(═O)NR₂, alkyl, alkenyl, cycloalkyl, heterocyclyl, and aryl, wherein each R above is independently selected from —H and an alkyl moiety, including, for example, C₁₋₆ alkyl (e.g., —CH₃, —C₂H₅, -isopropyl, -tert-butyl, etc.), C₁₋₆ alkoxy (e.g., —OCH₃, —OC₂H₅), halogenated C₁₋₆ alkyl (e.g., —CF₃, —CHF₂, —CH₂F), and halogenated C₁₋₆ alkoxy (e.g., —OCF₃, —OC₂F₅).

As used herein, the term “alkenyl” is intended to refer to linear or branched monovalent or divalent unsaturated hydrocarbon substituents, optionally substituted with one or more functional groups anywhere on or within the substituent. An alkenyl substituent can be viewed as being divalent if the sp² carbon is part of a molecule bearing the alkenyl substituent. An illustrative example is methylenecyclohexane, which can be viewed as cyclohexane substituted with a methylene group (i.e., a divalent alkenyl substituent, ═CH₂). Unless otherwise specified, an alkenyl group may contain any number of carbon atoms, such as for example, C₁-C₂₄, C₁-C₁₈, C₁-C₁₀, C₁-C₈, or C₁-C₆, and any degrees of unsaturation. Examples of alkenyl substituents include, but are not limited to, methylene/methylidine (═CH₂), ethylene/ethenyl (—CH═CH₂ or ═CH—CH₃), propylene/propenyl (—CH₂—CH═CH₂, cis or trans —CH═CH—CH₃, ═C(CH₃)₂, or cis or trans ═CH—CH₂CH₃), and so forth. An alkenyl substituent herein may be substituted, i.e., having one or more substituent groups appended on the alkenyl group or incorporated within the alkenyl chain. A substitution within the alkenyl substituent may include an ether, sulfide, or imine linkage, i.e., —O—, —S—, or —N═, for example, or some other intervening heteroatom(s). Examples of substitution on an alkenyl substituent include, but are not limited to, —CN, —N₃, —NH₂, —NHR, —N(R)₂, —N(R)₃⁺, —NO₂, —NH—NH₂, —NH—NHR, —NH—NR₂, -halo, —SH, —SR, —S(═O)R, —SO₂R, —OPO₃^{2—}, —PO₃^{2—}, —OH, —OR, —C(═O)R, —OC(═O)R, —CO₂R, —NHC(═O)R, —NRC(═O)R, —C(═O)NHR, —C(═O)NR₂, alkyl, alkenyl, cycloalkyl, heterocyclyl, and aryl, wherein each R above is independently selected from an alkyl moiety, including, for example, C₁₋₆ alkyl (e.g., —CH₃, —C₂H₅, -isopropyl, -tert-butyl, etc.), C₁₋₆ alkoxy (e.g., —OCH₃, —OC₂H₅), halogenated C₁₋₆ alkyl (e.g., —CF₃, —CHF₂, —CH₂F), and halogenated C₁₋₆ alkoxy (e.g., —OCF₃, —OC₂F₅).

As used herein, the term “aryl” includes any aromatic ring or fused polycyclic aromatic ring system, such as phenyl, naphthyl, anthracenyl, and phenanthrenyl, optionally substituted with one or more functional groups anywhere on the aromatic substituent. An unsubstituted phenyl substituent may also be denoted as —C₆H₅ or more simply, -Ph. Aromatic heterocyclic rings are distinct, and are included in the definition of heterocycyl substituents set forth below. Examples of substitution on an aryl substituent include, but are not limited to, —CN, —N₃, —NH₂, —NHR, —N(R)₂, —N(R)₃⁺, —NO₂, —NH—NH₂, —NH—NHR, —NH—NR₂, -halo, —SH, —SR, —S(═O)R, —SO₂R, —OPO₃^{2—}, —PO₃^{2—}, —OH, —OR, —C(═O)R, —OC(═O)R, —CO₂R, —NHC(═O)R, —NRC(═O)R, —C(═O)NHR, —C(═O)NR₂, alkyl, alkenyl, cycloalkyl, heterocyclyl, and aryl, wherein each R above is independently selected from an alkyl moiety, including, for example, C₁₋₆ alkyl (e.g., —CH₃, —C₂H₅, -isopropyl, -tert-butyl, etc.), C₁₋₆ alkoxy (e.g., —OCH₃, —OC₂H₅), halogenated C₁₋₆ alkyl (e.g., —CF₃, —CHF₂, —CH₂F), and halogenated C₁₋₆ alkoxy (e.g., —OCF₃, —OC₂F₅).

As used herein, “heterocycle” is intended to refer to an unsubstituted or optionally substituted, saturated, unsaturated or aromatic, carbocyclic ring interrupted in its carbocyclic structure by at least one heteroatom selected from oxygen (O), sulfur (S) or nitrogen (N). As used herein, the term “heterocyclyl” is intended to refer to a heterocycle as a substituent group, being attached to another atom in a compound from any C atom or heteroatom present in the heterocyclic ring. For example, “pyridinyl” includes 2-, 3- and 4-pyridinyl moieties as substituent groups. Heterocycles may be monocyclic or fused polycyclic in structure. Examples of optional substitution on an aryl substituent include, but are not limited to, —CN, —N₃, —NH₂, —NHR, —N(R)₂, —N(R)₃⁺, —NO₂, —NH—NH₂, —NH—NHR, —NH—NR₂, -halo, —SH, —SR, —S(═O)R, —SO₂R, —OPO₃^{2—}, —PO₃^{2—}, —OH, —OR, —C(═O)R, —OC(═O)R, —CO₂R, —NHC(═O)R, —NRC(═O)R, —C(═O)NHR, —C(═O)NR₂, alkyl, alkenyl, cycloalkyl, heterocyclyl, and aryl, wherein each R above is independently selected from an alkyl moiety, including, for example, C₁₋₆ alkyl (e.g., —CH₃, —C₂H₅, -isopropyl, -tert-butyl, etc.), C₁₋₆ alkoxy (e.g., —OCH₃, —OC₂H₅), halogenated C₁₋₆ alkyl (e.g., —CF₃, —CHF₂, —CH₂F), and halogenated C₁₋₆ alkoxy (e.g., —OCF₃, —OC₂F₅).

Examples of heterocycles include but are not limited to: azepinyl, aziridinyl, azetyl, azetidinyl, diazepinyl, dithiadiazinyl, dioxazepinyl, dioxolanyl, dithiazolyl, furanyl, isooxazolyl, isothiazolyl, imidazolyl, morpholinyl, morpholino, oxetanyl, oxadiazolyl, oxiranyl, oxazinyl, oxazolyl, piperazinyl, pyrazinyl, pyridazinyl, pyrimidinyl, piperidyl, piperidino, pyridyl, pyranyl, pyrazolyl, pyrrolyl, pyrrolidinyl, thiatriazolyl, tetrazolyl, thiadiazolyl, triazolyl, thiazolyl, thienyl, tetrazinyl, thiadiazinyl, triazinyl, thiazinyl, thiopyranyl furoisoxazolyl, imidazothiazolyl, thienoisothiazolyl, thienothiazolyl, imidazopyrazolyl, cyclopentapyrazolyl, pyrrolopyrrolyl, thienothienyl, thiadiazolopyrimidinyl, thiazolothiazinyl, thiazolopyrimidinyl, thiazolopyridinyl, oxazolopyrimidinyl, oxazolopyridyl, benzoxazolyl, benzisothiazolyl, benzothiazolyl, imidazopyrazinyl, purinyl, pyrazolopyrimidinyl, imidazopyridinyl, benzimidazolyl, indazolyl, benzoxathiolyl, benzodioxolyl, benzodithiolyl, indolizinyl, indolinyl, isoindolinyl, furopyrimidinyl, furopyridyl, benzofuranyl, isobenzofuranyl, thienopyrimidinyl, thienapyridyl, benzothienyl, cyclopentaoxazinyl, cyclopentafuranyl, benzoxazinyl, benzothiazinyl, quinazolinyl, naphthyridinyl, quinolinyl, isoquinolinyl, benzopyranyl, pyridopyridazinyl and pyridopyrimidinyl. Further examples of heterocyclic systems may be found in A. Katritzky, et al., Handbook of Heterocyclic Chemistry, 3^{rd} Ed., Elsevier, 2010, the entire contents of which are incorporated by reference herein.

### General Examples

In various examples of the present disclosure, novel polymeric nanogel particles are described. Various nanogel particles herein exhibit dual functionality through the presence of at least two types of reactive end groups on copolymer chains within the nanogel particles. For example, the present nanogel particles may exhibit temperature responsiveness wherein nanogel particles can shrink or swell in response to temperature changes, and pH responsiveness that assists in surface binding reactions. Nanogel particles in accordance with the present disclosure may, among other things, find use in nucleic acid sequencing methods, in particular within flow cells used in SBS methods.

In various examples, nanogel particles are prepared by a suspension/precipitation free radical polymerization of various monomer types. Nanogel particles herein are described by the synthetic processes used to prepare them, i.e., the monomers used in the suspension/precipitation free radical polymerization reaction and the reaction conditions, and also structurally, such as by describing certain recurring units present in copolymer chains of the nanogel particles thus prepared, along with physical properties. In various examples, recurring monomer units in a copolymer chain of a nanogel particle may include part of a block within a block copolymer.

In various examples, nanogel particles include copolymer chains that are crosslinked. Crosslinking is expected, for example, if a multifunctional monomer is used in the suspension/precipitation free radical polymerization along with other monomer types.

In various examples, incorporating monomers resulting in temperature or pH responsive nanogel particles, the nanogel particle sizes can be fine-tuned to be adaptable with any step in SBS sequencing protocol, such as, library seeding, nanogel particle capture in nano-wells of a FC, clustering on particles, and sequencing on particles. In various examples, temperature responsiveness can be incorporated using LCST (lower critical solution temperature) or UCST (upper critical solution temperature).

Monomers for Nanogel Particle Synthesis

In various examples, a first type of monomer for use in synthesizing nanogel particles in a suspension/precipitation free radical polymerization reaction include species having a structure:

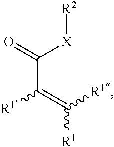

wherein:
each of R¹, R^{1′}, and R^{1″} is independently selected from H, halogen, alkyl, alkoxy, alkenyl, alkynyl, cycloalkyl, aryl, heteroaryl, or heterocyclyl;
X is —O— or —NH—; and
R² is —CH₂—C≡CH, or R² has a structure:

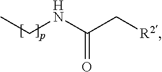

wherein R^{2′} is —N₃ or —C≡CH; and
p is an integer of 1 to 50.

Examples of this first type of monomer include, but are not limited to, propargyl acrylate, N-propargyl acrylamide, N-(5-(2-azidoacetamido)pentyl)acrylamide, (2-methacryloyloxy)trimethylammonium chloride, 2-acrylamido-2-methyl-1-propanesulfonic acid, [2-(acryloyloxy)ethyl]trimethylammonium chloride, and 2-hydroxyethylmethacrylate.

In various examples, a second type of monomer for use in synthesizing nanogel particles in a suspension/precipitation free radical polymerization reaction include species having a structure:

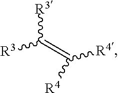

wherein:
each of R³, R^{3′}, R⁴, and R^{4′} is independently selected from —H, —R⁵, —OR⁵, —CO₂R⁵, —C(O)R⁵, —OC(O)R⁵, —C(O)NR⁶R⁷, and —NR⁶R⁷; R⁵ is —H, —OH, alkyl, cycloalkyl, hydroxyalkyl, aryl, heteroaryl, or heterocyclyl; and each of R⁶ and R⁷ is independently selected from —H and alkyl.

Examples of this second type of monomer include, but are not limited to, acrylic acid, methacrylic acid, acrylamide, methacrylamide, N-isopropylacrylamide, N-isopropylmethacrylamide, N,N-dimethylacrylamide, N,N-dimethylmethacrylamide, N-vinylpyrrolidone, and N-vinylpyridine.

In various examples, nanogel particles are prepared under suspension/precipitation free radical polymerization reaction conditions by reacting at least one first type of monomer and at least one second type of monomer, in accordance with the above recited structures. With these two types of monomers used in a suspension/precipitation free radical polymerization reaction, resulting nanogel particles include copolymer chains having at least a first repeating unit incorporating the first type of monomer and at least a second repeating unit incorporating the second type of monomer.

Multifunctional monomers that can be included in a suspension/precipitation polymerization reaction to form nanogel particles having some degree of crosslinking between copolymer chains include, but are not limited to, N,N′-methylenebisacrylamide, N,N′-methylenebismethacrylamide, polyethylene glycol diacrylate, polyethylene glycol dimethacrylate, N-vinylacrylamnide, glycidyl acrylate, divinylbenzene, diallyldimethylammonium chloride, and tetraallyl ammonium chloride.

In various examples, nanogel particles are prepared under suspension/precipitation free radical polymerization reaction conditions by reacting at least one first type of monomer, at least one second type of monomer, both in accordance with the above recited structures, and at least one multifunctional monomer. With these two types of monomers and a multifunctional monomer used in a suspension/precipitation free radical polymerization reaction, resulting nanogel particles include copolymer chains having at least a first repeating unit incorporating the first type of monomer and at least a second repeating unit incorporating the second type of monomer, wherein the copolymer chains have at least some degree of crosslinking between copolymer chains.

In various examples, nanogel particles are prepared under suspension/precipitation free radical polymerization reaction conditions by reacting at least one first type of monomer, at least one second type of monomer, both in accordance with the above recited structures, and the multifunctional monomer N,N′-methylenebismethacrylamide (BisAM).

In various examples, nanogel particles thus prepared under suspension/precipitation free radical polymerization reaction conditions including at least one of each of two types of monomers and optionally a multifunctional monomer as described above include copolymer chains including at least one carboxylic acid end group (i.e., the monovalent substituent —CO₂— or —CO₂H) and copolymer chains including at least one —N₃ or —C≡CH end group.

Suspension/Precipitation and Other Free-Radical Polymerizations

In various examples, syntheses of nanogel particles include various aspects of suspension/precipitation free-radical polymerization or emulsion polymerization. In various embodiments, reaction conditions are aqueous and heated, employing selected monomers, a dispersant to facilitate suspension of generally water-insoluble nanogel particles thus formed in water, and a free-radical initiator.

In various examples, a suspension/precipitation free-radical polymerization reaction is conducted at a temperature of from about 50° C. to about 90° C., over the course of about 1 hour to 4 hours.

In various examples, a dispersant herein comprises an anionic or nonionic dispersant. Exemplary anionic dispersants included sodium dodecyl sulfate (SDS). Nonionic dispersants include, but are not limited to, polyethylene glycol (PEG), sorbitan monooleates (e.g., under the brand name Span®), ethoxylated sorbitan monooleates (e.g., under the brand name Tween®), and acryloyl-terminated PEG

In various examples, a free-radical initiator includes a water-soluble compound.

In various examples, a free-radical initiator includes a peroxide.

In various examples, a free-radical initiator includes sodium, potassium, or ammonium persulfate.

In various examples, a free-radical initiator includes ammonium persulfate (APS).

In various examples, nanogel particles are synthesized in a suspension/precipitation free-radical polymerization reaction incorporating a dispersed monomer mixture including N-(5-(2-azidoacetamido)pentyl)acrylamide (AzAPA); N-isopropylacrylamide (NiPAM); and acrylic acid (AAc).

In various examples, nanogel particles are synthesized in a suspension/precipitation free-radical polymerization reaction incorporating a dispersed monomer mixture including N-(5-(2-azidoacetamido)pentyl)acrylamide (AzAPA); N-isopropylacrylamide (NiPAM); and acrylic acid (AAc); and the multifunctional monomer N,N′-methylenebisacrylamide (BisAM).

In various examples, nanogel particles are synthesized in a suspension/precipitation free-radical polymerization reaction incorporating a dispersed monomer mixture including propargyl acrylate (PAG); N-isopropylacrylamide (NiPAM); and acrylic acid (AAc).

In various examples, nanogel particles are synthesized in a suspension/precipitation free-radical polymerization reaction incorporating a dispersed monomer mixture including propargyl acrylate (PAG); N-isopropylacrylamide (NiPAM); and acrylic acid (AAc); and the multifunctional monomer N,N′-methylenebisacrylamide (BisAM).

In various examples, nanogel particles are synthesized in a suspension/precipitation free-radical polymerization reaction incorporating a dispersed monomer mixture including propargyl acrylamide (PAM); N-isopropylacrylamide (NiPAM); and acrylic acid (AAc).

In various examples, nanogel particles are synthesized in a suspension/precipitation free-radical polymerization reaction incorporating a dispersed monomer mixture including propargyl acrylamide (PAM); N-isopropylacrylamide (NiPAM); and acrylic acid (AAc); and the multifunctional monomer N,N′-methylenebisacrylamide (BisAM).

Nanogel Particles Including Copolymer Chains

In various examples, nanogel particles include copolymer chains having various end groups on at least some of the copolymer chains. In various examples, nanogel particles include copolymer chains having at least some degree of crosslinking.

In various examples, polymeric nanogel particles in accordance with the present disclosure include copolymer chains further including a first recurring unit of Formula (I):

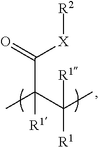

wherein:
each of R¹, R^{1′}, and R^{1″} is independently selected from H, halogen, alkyl, alkoxy, alkenyl, alkynyl, cycloalkyl, aryl, heteroaryl, or heterocyclyl;
X is —O— or —NH—; and
R² is —CH₂—C≡CH, or R² has a structure:

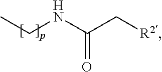

wherein R^{2′} is —N₃ or —C≡CH; and
p is an integer of 1 to 50; and
a second recurring unit of Formula (II),

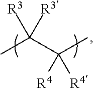

wherein:
each of R³, R^{3′}, R⁴, and R^{4′} is independently selected from —H, —R⁵, —OR⁵, —CO₂R⁵, —C(O)R⁵, —OC(O)R⁵, —C(O)NR⁶R⁷, and —NR⁶R⁷; wherein R⁵ is —H, —OH, alkyl, cycloalkyl, hydroxyalkyl, aryl, heteroaryl, or heterocyclyl; and each of R⁶ and R⁷ is independently selected from —H and alkyl.

In various examples, R¹═R^{1′}═R^{1″}═H; X is —O— or —NH—; R² is —CH₂—C≡CH or has a structure:

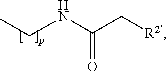

wherein R^{2′} is —N₃ or —C≡CH; and p is an integer of 1 to 50.

In various examples, nanogel particles having the above-recited recurring units include copolymer chains having at least one carboxylic acid end group.

In various examples, nanogel particles having the above-recited recurring units include copolymer chains having at least one —N₃ or —C≡CH end group.

In various examples, nanogel particles include copolymer chains wherein the first recurring unit of Formula (I) is:

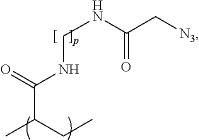

wherein p, as per above, is an integer of 1 to 50.

In various examples, nanogel particles include copolymer chains wherein the first recurring unit of Formula (I) is:

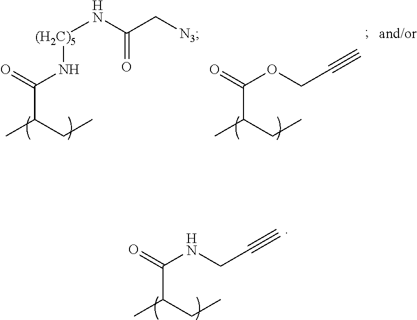

In various examples, nanogel particles include copolymer chains wherein the second recurring unit of Formula (II) is at least one of:

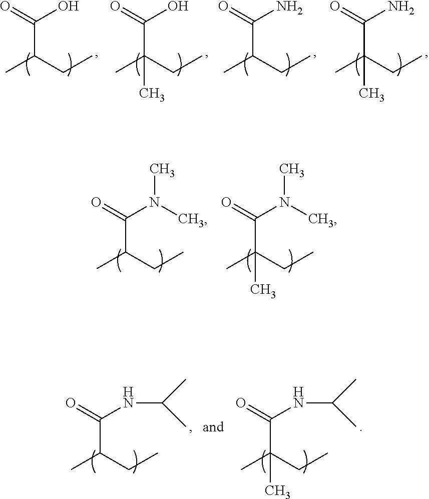

In various examples, polymeric nanogel particles include poly(AzAPA-co-NiPAM-co-AAc-co-BisAM) copolymer chains. In various examples, at least some of these copolymer chains include —N₃ end groups and —CO₂H end groups for dual functionality.

In various examples, polymeric nanogel particles include poly (PAG-co-NiPAM-co-AAc-co-BisAM) copolymer chains. In various examples, at least some of these copolymer chains include —C≡CH end groups and —CO₂H end groups.

In various examples, polymeric nanogel particles include poly (PAM-co-NiPAM-co-AAc-co-BisAM) copolymer chains. In various examples, at least some of these copolymer chains include —C≡CH end groups and —CO₂H end groups for dual functionality.

Grafting Amplification Primers onto Nanogel Particles

The above general examples show that nanogel particles can be prepared wherein copolymer chains of the nanogel particles include at least some —CO₂H end groups and at least some —N₃ end groups or —C≡CH end groups. In some examples, the —CO₂H end groups of the copolymer chains are leveraged in attaching nanogel particles to surfaces such as lanes within a FC used for SBS. Further, the —N₃ end groups or —C≡CH end groups of the copolymer chains may be leveraged in grafting suitably functionalized amplification primers (such as P5/P7) onto each nanogel particle in preparation for SBS.

In various examples, grafting of an amplification primer to a polymeric nanogel particle includes click-chemistry between a terminal alkyne substituent on the amplification primer and an —N₃ end group of a respective copolymer chain, or click-chemistry between a terminal —N₃ substituent on the amplification primer and a —C≡CH end group on a respective copolymer chain. Stated another way, grafting of amplification primers to nanogel polymers in various examples includes alkyne-azide or azide-alkyne cycloaddition click chemistry, covalently linking primer to nanogel particle through a triazine moiety.

In other aspects, thiol-functionalized primers may be grafted onto nanogel particles including copolymer chains wherein at least some of the copolymer chains include —C≡CH end groups.

In various examples, and as detailed above, the choice of monomers used in the synthesis of the nanogel particles dictates whether the resulting copolymer chains include —N₃ or —C≡CH end groups. For grafting, a complementary functional group is chosen for the functionalized amplification primer to promote click-chemistry.

P5 and P7 amplification primers for use herein are used on the surface of commercial flow cells sold by Illumina Inc. for sequencing on the HiSeq™, MiSeq™, NextSeq™ and Genome Analyzer™ platforms. P5/P7 amplification primers for grafting onto nanogel particles are fully described in U.S. Pat. No. 9,982,250 and U.S. Publication No. 2011/0059865, the disclosures of which are incorporated herein by reference in their entireties.

In various examples, functionalized amplification primers for grafting onto nanogel particles include, but are not limited to, alkyne-P5/P7 primers, N₃-P5/P7 primers, and thiol-P5/P7 primers.

In various examples, grafting of alkyne-P5/P7 primers onto nanogel particles that include copolymer chains having —N₃ end groups includes CuAAC grafting, resulting in P5/P7-grafted nanogel particles, such as ANA-P5P7 grafted nanogel particles.

In various examples, CUAAC catalyzed click chemistry involving N₃-P5/P7 or thiol-P5/P7 primers is conducted at a temperature of from about 40° C. to about 80° C., and from about 1 hour to about 5 hours.

In various examples, grafting of N₃-P5/P7 or thiol-P5/P7 primers onto nanogel particles that include copolymer chains having —C≡CH end groups includes CuAAC Blackpool grafting, resulting in P5/P7-grafted nanogel particles, such as PANA-P5P7 or PANA′-P5P7 grafted nanogel particles.

Capture of Primer-Grafted Nanogel Particles for SBS

In various examples, and as part of an SBS method, primer-grafted nanogel particles, such as ANA-P5P7, PANA-P5P7, or PANA′-P5P7 primer-grafted nanogel particles, are captured on surfaces in a flow cell (FC) such as the HiSeq™ FC from Illumina, Inc. Primer-grafted nanogel particles may be captured into nano-wells patterned in coatings on the FC surface, or directly attached to coatings on surfaces absent nano-wells. In various examples, each primer-grafted nanogel particle can act as a nano-well and thus can function as a replacement for the same.

In various examples, primer-grafted nanogel particles are captured into nano-wells of a FC by either:
(a) a bioconjugation technique using DMTMM to activate reaction between the free carboxylic acid end groups present on the copolymer chains of the primer-grafted nanogel particles and —NH₂ groups available on a previously silanized FC surface to form amide linkages, or
(b) CuAAC click-chemistry to react any remaining —C≡CH end groups still present on copolymer chains of the primer-grafted nanogel particles (i.e., after grafting) with reactive —N₃ groups present on a standard PAZAM coated and polished FC to form triazine linkages.

In various examples relating to (a) above, silanization of a FC surface may be accomplished using any suitable silane or silane derivative. The method used to attach a silane or silane derivative to a substrate may vary depending upon the silane or silane derivative that is being used.

In various examples, the silane or silane derivative is 3-aminopropyltriethoxysilane (APTES) or 3-aminopropyltrimethoxysilane (APTMS) (i.e., silanes having the general structure, X—R^{B}—Si(OR^{C})₃, wherein X is amino, R^{B} is —(CH₂)₃—, and R^{C} is ethyl or methyl). In this example, a FC surface may be pre-treated with APTES or APTMS to covalently link silicon to one or more oxygen atoms on the surface. This chemically treated surface is optionally baked to form an amine group monolayer.

In various examples, the plurality of —NH₂ groups present on a FC surface are then reacted with carboxylic acid end groups present on corresponding copolymer chains of the primer-grafted nanogel particles. This procedure takes advantage of the dual functionality of the nanogel particles in that the —CO₂H end groups are only used to bind the nanogel particles to the FC surfaces while either —N₃ or —C≡CH end groups were previously used only for grafting functionalized primers to the nanogel particles.

In various examples relating to (b) above, PAZAM coatings on FC surfaces are prepared using N-(5-(2-bromoacedamido)pentyl)acrylamide (BrAPA) as a monomer for polymeric hydrogel coating, followed by conversion of the bromo groups to —N₃ groups.

In various examples, PAZAM may be deposited on the surface of a patterned FC surface by spin coating, dipping, dip coating, or flow of the PAZAM under positive or negative pressure, or another suitable technique. The PAZAM may be present in a mixture. In an example, the mixture includes PAZAM in water or in an ethanol and water mixture.

After being coated, the functionalized molecule may also be exposed to a curing process to form the functionalized coating layer across the entire patterned substrate (i.e., on depression(s) and interstitial region(s)). In an example, curing the functionalized molecule may take place at a temperature ranging from room temperature (e.g., about 25° C.) to about 60° C. for a time ranging from about 5 minutes to about 2 hours.

To form a PAZAM coating layer in nano-wells and not on interstitial regions of a patterned substrate, the PAZAM coating layer may be polished off of the interstitial regions using either (a) a basic, aqueous slurry having a pH ranging from about 7.5 to about 11 and including an abrasive particle, or (b) a polishing pad and a solution free of an abrasive particle.

To capture nanogel particles onto PAZAM coated FC surfaces, the PAZAM coating having reactive —N₃ groups can react with any remaining —C≡CH end groups present on copolymer chains of the nanogel particles under conditions for CuAAC click-chemistry. For any additional details, see the '033 Patent (Illumina) referenced above and incorporated herein.

In alternative examples, the order of the distinct steps of primer grafting and particle capture can be reversed. Thus, nanogel particles having dual functionality may be captured on silanized FC surfaces or PAZAM coated FC surfaces by amide formation or click-chemistry, and then the captured particles are subsequently exposed to suitably functionalized amplification primers (alkyne-P5/P7 or N₃-P5/P7, for example) to append the amplification primers to the captured nanogel particles.

Seeding, Clustering and SBS Sequencing

In various examples, clustering includes either in-suspension clustering or on-board clustering. On-board clustering may be used for proof-of-concept since suspension clustering avoids the need for patterning coated FC surfaces and each nanogel particle captured on the FC surface functions as its own nano-well. In suspension clustering, seeded ssDNA may be clustered on the surfaces of the nanogel particles. Clustering on nanogel particles is dependent on having sufficiently accessible primers grafted onto the nanogel particles.

In various examples, the temperature responsiveness of primer-grafted nanogel particles having blocks of poly(NiPAM) within copolymer chains, allows for temperature-controlled manipulation of seeding, amplification, and sequencing by:
(a) promoting temperature-controlled shrinkage during seeding to reduce the probability for multiple seeding events and therefore enhance monoclonality;
(b) promoting temperature-controlled swelling during clustering to increase primer accessibility and facilitate diffusion of materials into polymeric nanogel particles, resulting in an increased number of strands per cluster/particles and improvement its fluorescent; and/or
(c) promoting temperature-controlled shrinking or swelling to improve the SBS steps of incorporation and cleavage.

In various examples, FCs having captured primer-grafted nanogel particles are then used in a variety of sequencing approaches or technologies, including SBS, cyclic-array sequencing, sequencing-by-ligation, pyrosequencing, and so forth. With any of these techniques, since the sequencing primers are present only on the nanogel particles, amplification will be confined to each particle. Moreover, due to confinement of amplification to particle surfaces, there is more time to amplify one sequencing template into larger clusters.

In various examples, SBS may be run on a system such as the HISEQ™, HISEQX™ MISEQ™, NOVASEQ™, or NEXTSEQ™ sequencer systems (Illumina, Inc.). In SBS, extension of a nucleic acid primer along a nucleic acid template (i.e., the sequencing template) is monitored to determine the sequence of nucleotides in the template. The underlying chemical process can be polymerization (e.g., catalyzed by a polymerase enzyme) or ligation (e.g., catalyzed by a ligase enzyme). In various polymerase based SBS processes, fluorescently labeled nucleotides are added to the primer to extend the primer in a template dependent fashion such that detection of the order and type of nucleotides added to the primer can be used to determine the sequence of the template. For example, to initiate a first SBS cycle, one or more labeled nucleotides, DNA polymerase, etc., may be delivered into/through a flow channel in the FC that houses an array of primers on nanogel particles. The primer-grafted nanogel particles whereupon primer extension causes a labeled nucleotide to be incorporated, can be detected through an imaging event. During an imaging event, an illumination system provides an excitation light to the nanogel particles.

In various examples, nucleotides can further include a reversible termination property that terminates further primer extension once a nucleotide has been added to the primer. For example, a nucleotide analog having a reversible terminator moiety can be added to the primer such that subsequent extension cannot occur until a deblocking agent is delivered to remove the moiety. Thus, for examples that use reversible termination, a deblocking reagent can be delivered to a flow channel before or after detection.

Flushes (i.e., washes) may take place between the various fluid delivery steps. The SBS cycle can then be repeated n times to extend the primer by n nucleotides, thereby detecting a sequence of length n.

To further illustrate the present disclosure, examples are provided below. These examples are provided for illustrative purposes and are not to be construed as limiting the scope of the disclosure in any way.

### EXAMPLES

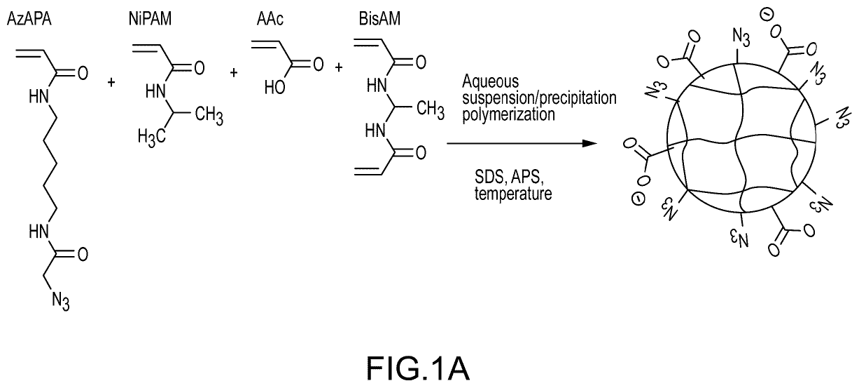

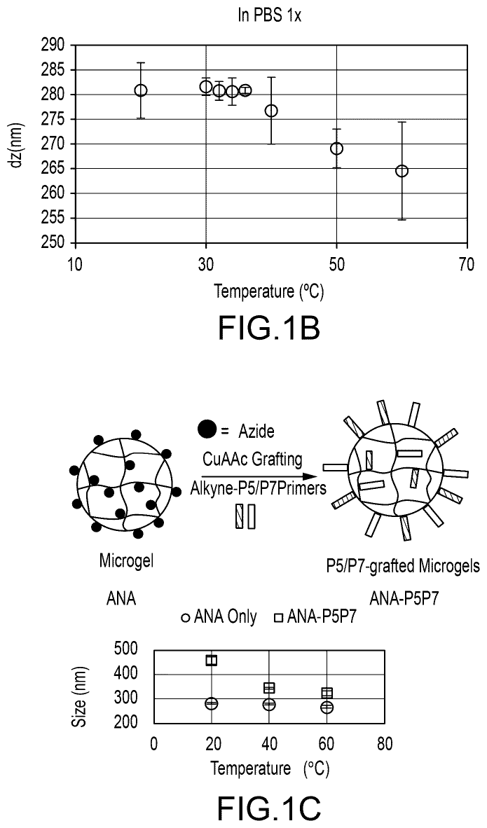

With reference now to FIG. 1A, an example procedure for preparing amide-based dual responsive nanogel particles is schematically illustrated in accordance with various examples of the present disclosure. As shown, the monomers N-(5-(2-azidoacetamido)pentyl)acrylamide (AzAPA), N-isopropylacrylamide (NiPAM), acrylic acid (AAc), and N,N′-methylenebisacrylamide (BisAM), are reacted under aqueous suspension/precipitation polymerization conditions to form nanogel particles having poly (AzAPA-co-NiPAM-co-AAc-co-BisAM) or “ANA” copolymer chains. In various examples, these particles include copolymer chains wherein at least some of the copolymer chains include at least one carboxylic acid end group and at least some of the copolymer chains include at least one —N₃ end group.

In various examples, the carboxylic acid end groups can be used to bind the nanogel particles to surfaces or to conjugate certain groups to the nanogel particles, and likewise the —N₃ end groups can be used to bind the nanogel particles to surfaces or to conjugate certain groups to the nanogel particles. In various examples, the nanogel particles are attached to flow cell surfaces through the free carboxylic acid end groups and/or the free —N₃ end groups.

The reaction scheme of FIG. 1A includes a suspension/precipitation polymerization reaction using SDS as dispersant and APS as free-radical polymerization initiator, with the reaction conducted in water and at, for example, 70° C. for 4-hours.

FIG. 1B sets forth a plot showing example nanogel particle size as a function of polymerization reaction temperature for the example nanogel particles produced in accordance with the reaction scheme of FIG. 1A. In general, the aqueous suspension/precipitation polymerization reaction depicted in FIG. 1A has proven to be well controlled, and desired nanogel particle sizes can be achieved with narrow polydispersity of the nanoparticles. For example, in sodium phosphate buffer (pH 7.4) the size ranges from 280 nm when the reaction is conducted at 20° C. to 265 nm when the reaction is conducted at 60° C. As shown, the Z-average particle size is relatively constant at about 280 nm when the suspension/precipitation polymerization reaction is run at less than about 40° C. As shown in the plot, above 40° C., (at which temperature polyNiPAM becomes hydrophobic), the nanogel particle size decreases per each degree higher than about 40° C. The vertical bars in the plot of FIG. 1B represent error bars.

FIG. 1C illustrates grafting functionalized amplification primers onto the nanogel particles including poly(AzAPA-co-NiPAM-co-AAc-co-BisAM) or “ANA” copolymer chains. In this example, the free —N₃ end groups present on at least some of the copolymer chains are used in click-chemistry grafting of alkyne-functionalized amplification primers to the free —N₃ end groups with formation of triazine linkages between particle and primer. The alkyne-functionalized amplification primers used here are functionalized P5/P7 primers and are thoroughly discussed in at least U.S. Pat. Nos. 9,815,916 and 10,266,891 (Illumina, Inc.), both incorporated herein by reference in their entireties.

With continued reference to FIG. 1C, nanogel particles having ANA copolymer chains with free —N₃ end groups, such as prepared in accordance with the reaction scheme set forth in FIG. 1A, were reacted with alkyne-functionalized P5/P7 amplification primers in suspension for 3 hours at 60° C. using copper-catalyzed azide-alkyne cycloaddition (CuAAC) click-chemistry to form P5/P7-grafted nanogel particles labeled in the FIG. 1C as “ANA-P5P7.” In ANA-P5P7 nanogel particles, free —N₃ end groups reacted with alkyne-functionalized primers to form triazine linkages linking amplification primer to particle.

The plot illustrated below the CuAAc grafting reaction in FIG. 1C shows the temperature dependent size of the ANA-P5P7 grafted nanogel particles. As shown, the ANA-P5P7 grafted nanogel particles shrink with increasing temperature.

In various examples, this temperature controllable shrinkage and swelling of nanogel particles such as ANA-P5P7 shown in FIG. 1C is a significant advantage. This characteristic can be used to increase the efficiency of various biochemical processes occurring during seeding, amplification, and nucleic acid sequencing. For example, shrinkage of particles can be promoted during seeding to reduce the probability for multiple seeding events and thus enhance monoclonality. Also, swelling of particles can be promoted during clustering to increase primer accessibility and facilitate diffusion of polymer, resulting in an increased number of nucleic acid strands per cluster of particles, subsequently improving fluorescent signal. Further, SBS steps such as incorporation or cleavage can also be improved by this observed opportunistic swelling/shrinking of amplification primer grafted nanogel particles.

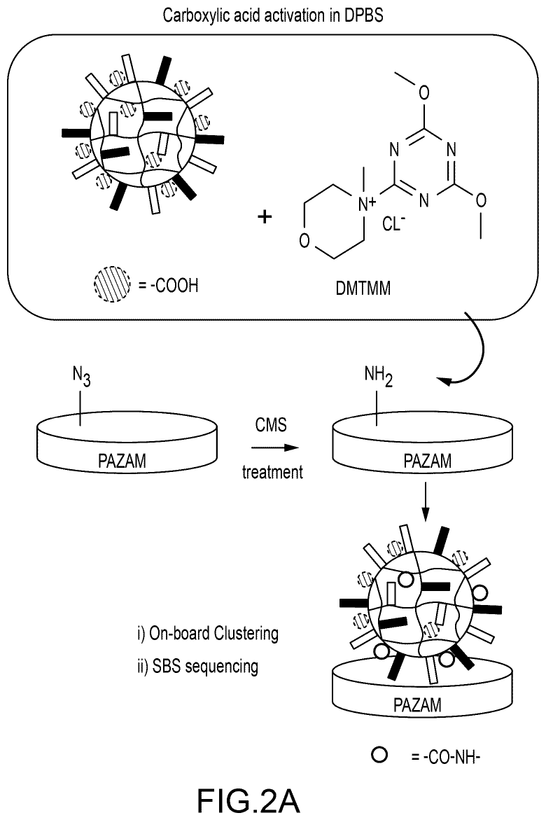

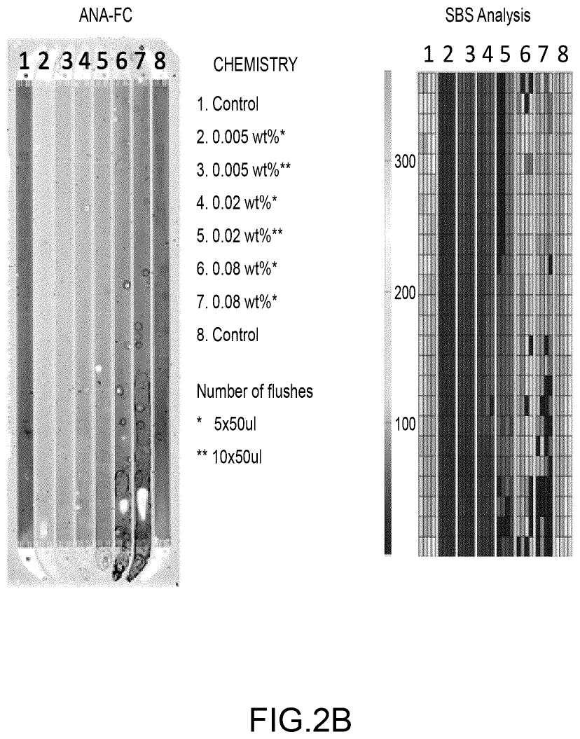

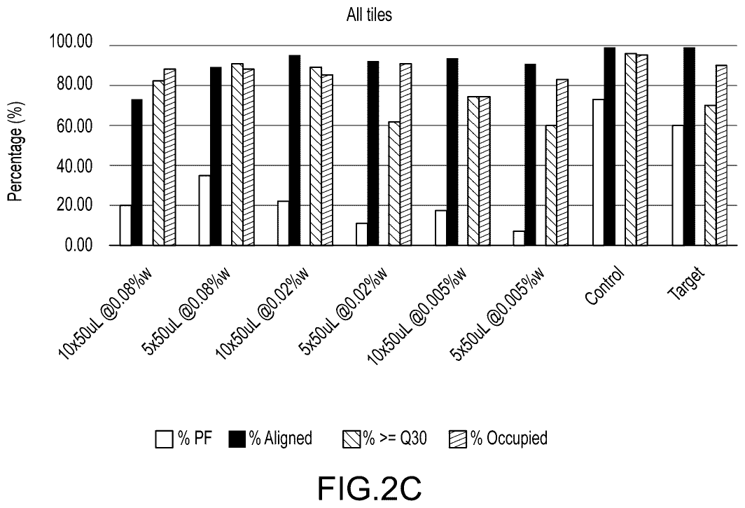

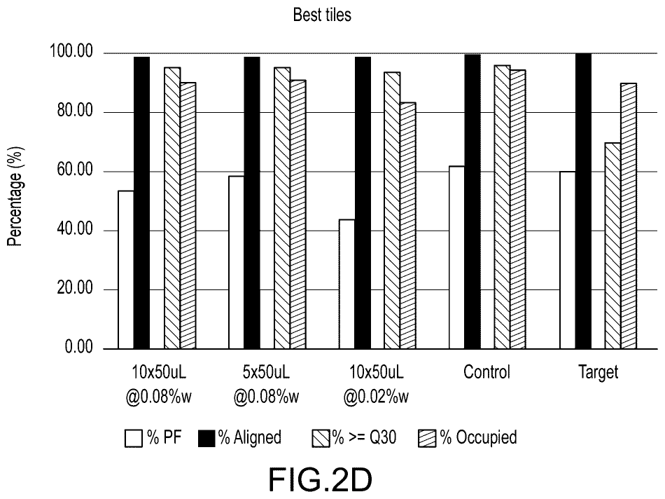

FIGS. 2A-2D show an example of chemically promoted capture chemistry of the ANA-P5P7 particles into the nano-wells of a FC and SBS sequencing metrics of the particle-captured FC.

FIG. 2A shows the bioconjugation technique using 4-(4,6-dimethoxy-1,3,5-triazin-2-yl)-4-methylmorpholinium chloride (DMTMM) used to bind the ANA-P5P7 particles to the nano-well, including reacting the free carboxylic acid end groups on the copolymer chains of the ANA-P5P7 particles with amine groups provided on the cleavage-mix-treated (CMS) nano-well. The reaction was conducted in PBS buffer (pH 7.4). The particles were then captured on a standard PAZAM coated and polished HiSeqX™ FC (P.N. 15059930) using a “cBot” (an automated fluidic device from Illumina used to prepare HiSeq X flow cells for cluster generation. See https://www.illumina.com/products/by-type/accessory-products/cbot.html). Different number of flushes and/or concentration of the particle suspension (wt. %) were investigated to optimize the capture process.

FIG. 2B (left image) shows a Cal Fluor Red (CFR) fluorescence image of the FC with ANA-P5P7 particles captured onto its surface, as imaged by the Typhoon™ at 580 nm. P5/P7 complimentary primers with attached CFR dye were hybridized on each lane. Lanes 1 and 8 are the control lanes, where the pristine PAZAM coated lanes are grafted with P5/P7 primers. As seen in FIG. 2B (left image), successful capture was achieved with good intensity of the hybridized CFR imaged by the Typhoon™. This experiment proved the ANA-P5P7 nanogel particles can accommodate a surface primer density compatible with clustering and sequencing. This FC was then clustered and sequenced using Illumina's HiSeqX™ instrument. The analyzed image of the intensity of the FC after sequencing is shown in FIG. 2B (right image).

Particle concentration and number of flushes of the particle solution are essential for intensity measured by CFR-QC or C1*Cycle 1 intensity using the sequencer. Three different particle concentrations were investigated, namely 0.08, 0.02, and 0.005 wt. %, with 5 or 10 flushes through each lane. Both CFR and C1 intensities were seen to increase with higher particle concentration and number of flushes.

FIGS. 2C and 2D show sequencing metrics where the average values of the best tiles and of all tiles, respectively, are considered.

The target values for a successful SBS set of metrics, including % PF, % Alignment, %≥Q30 and % Occupancy were: 60, 100, 70 and 90%, respectively. In all experiments, three different particle concentrations were investigated, namely 0.08, 0.02, and 0.005 wt. %, with 5 or 10 flushes though each lane, as shown in FIG. 2B (under “chemistry”). The average values of all metrics, compared to those of the control lanes and of the target values, demonstrate successful SBS sequencing after 36 cycles. More specifically, the average metric values of the best tiles (FIG. 2C) clearly show that these values are highly achieved. In particular, for a particle concentration of 0.08 wt. % and 5 flushes through the lane, % PF, % Alignment, %≥Q30 and % Occupancy were 58.2, 99.4, 95.1 and 90.5%, respectively. FIG. 2D is a bar chart setting forth each of the metrics for just the best tiles. The results obtained thus demonstrate the ability of ANA-P5P7 nanogel particles to support successful SBS sequencing.

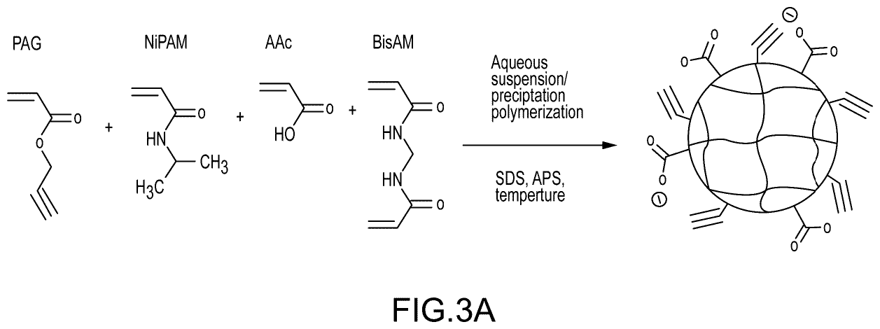

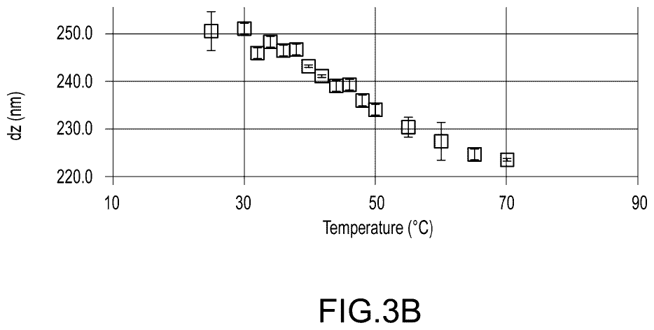

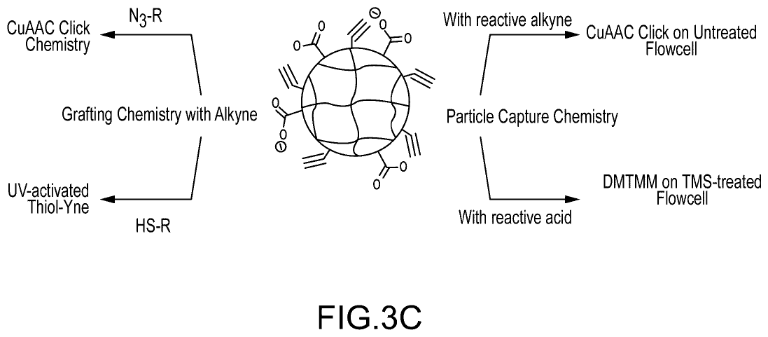

As illustrated in FIGS. 3A-3C, amide-based dual responsive nanogel particles can also be synthesized by reacting propargyl acrylate (PAG) or propargyl acrylamide (PAM), N-isopropylacrylamide (NiPAM), acrylic acid (AAc), and N,N′-methylenebisacrylamide (BisAM) monomers under aqueous suspension/precipitation polymerization conditions to form nanogel particles having poly (PAG-co-NiPAM-co-AAc-co-BisAM) “PANA” or poly (PAM-co-NiPAM-co-AAc-co-BisAM) “PANA”′ copolymer chains. In various examples, these particles include copolymer chains wherein at least some of the copolymer chains include at least one carboxylic acid end group and at least some of the copolymer chains include at least one —C≡CH end group. In various examples, the carboxylic acid end groups can be used to bind the nanogel particles to FC surfaces or to conjugate certain groups to the nanogel particles. The —C≡CH end groups can be used to bind the nanogel particles to FC surfaces or to graft certain groups such as primers to the nanogel particles. In various examples, the nanogel particles are attached to FC surfaces through the free carboxylic acid end groups or the free —C≡CH end groups.

In the synthesis example shown in FIG. 3A, propargyl acrylate (PAG), N-isopropylacrylamide (NiPAM), acrylic acid (AAc), and N,N′-methylenebisacrylamide (BisAM) monomers were reacted under aqueous suspension/precipitation polymerization conditions at 70° C. for 4-hours and in the presence of SDS and APS to form nanogel particles having poly(PAG-co-NiPAM-co-AAc-co-BisAM) or PANA copolymer chains. At least some of the polymer chains include carboxylic acid end groups and at least some of the copolymer chains include —C≡CH end groups.

As shown in the plot in FIG. 3B, the resulting PANA nanogel particles exhibited temperature dependent shrinkage/swelling, ranging from a swollen size of about 250 nm at temperatures less than about 30° C. down to s shrunken size of about 225 nm at temperatures around 70° C. (determined in pH 7.4 phosphate buffer, using light scattering). Further, at temperatures above about 40° C., the temperature at which poly(NiPAM), present as blocks in the block copolymer chains, becomes hydrophobic, the particle size decreased more significantly with increasing temperature.

FIG. 3C schematically illustrates the various reactions subsequently conducted on the PANA nanogel particles produced in accordance with FIG. 3A. Namely, PANA nanogel particles were grafted with N₃-P5/P7 primers in a reaction using copper based catalyzed click chemistry (CuAAC) and conducted for 3 hours at 60° C. Primer-grafted PANA particles are purified by TFF and isolated. The primer density thus obtained appeared to be compatible with SBS requirements based on CFR QC. Alternatively, the grafting can be accomplished using thiol-P5/P7 primers in a UV-activated thiol-alkyne click-chemistry reaction, as shown in FIG. 3C.

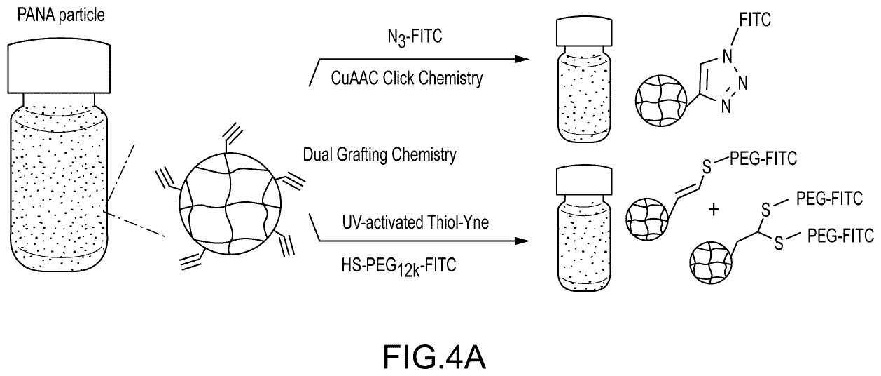

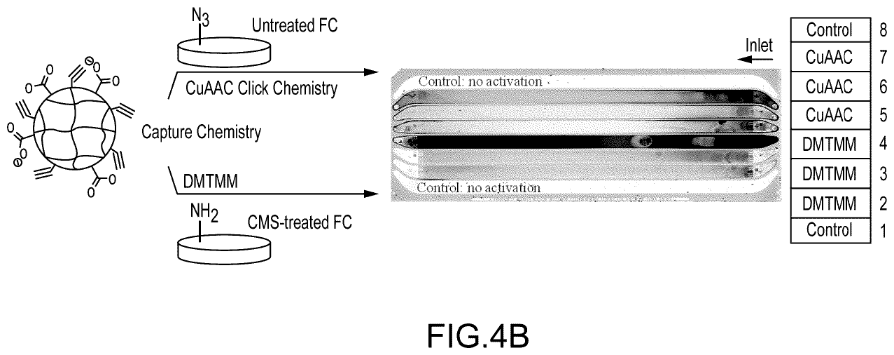

FIG. 4A schematically illustrates example click-chemistry reactions of FITC dye to —C≡CH end groups present on the nanogel particles. This is done via N₃-FITC utilizing CuAAC chemistry. Alternatively, UV-activated thiol-alkyne chemistry can be utilized for the click-chemistry reaction between HS-PEG₁₂ₖ-FITC and PANA particles.

FIG. 4B schematically illustrates PANA particle capture. Having both —C≡CH and carboxylic acid functional groups on the PANA particles, two different capture chemistries can be employed. The PANA particles can be captured into nano-wells of a FC via CuAAC or DMTMM bioconjugation to CMS treated PAZAM. The particles are captured on a standard PAZAM coated and polished HiSeqX™ FC (P.N. 15059930) using a cBot. Different number of flushes of the same particle concentration (wt. %) were investigated to optimize the capture process.

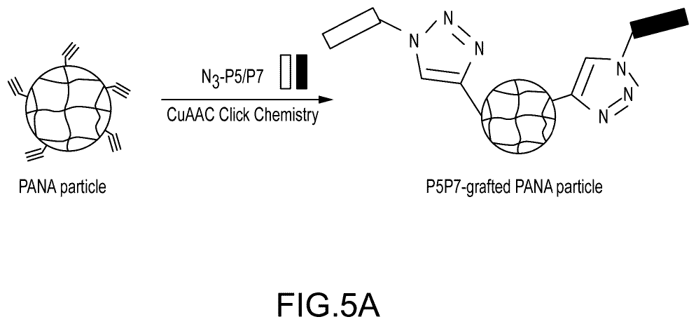

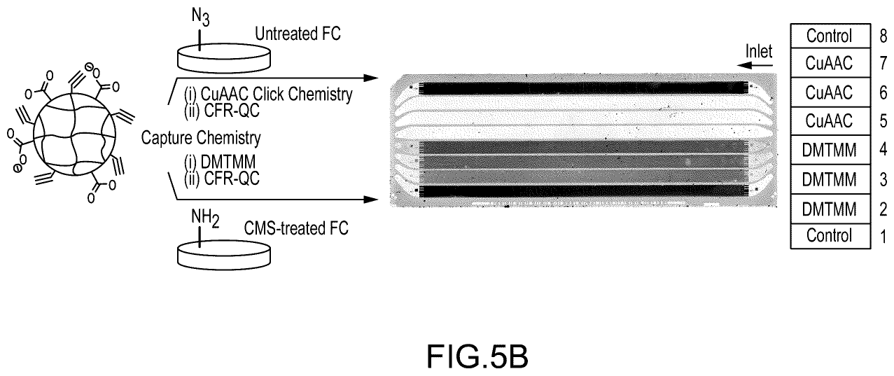

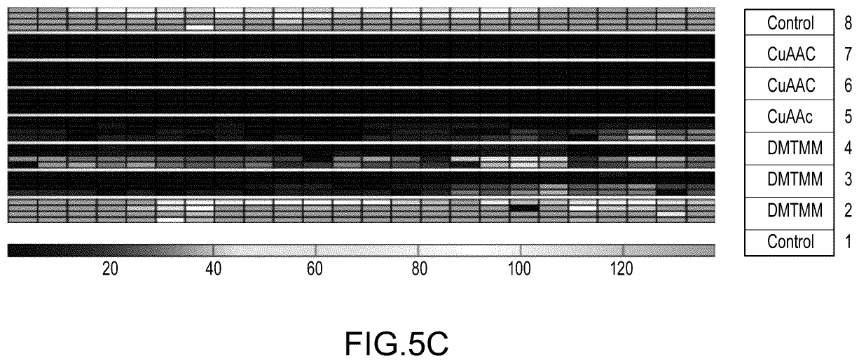

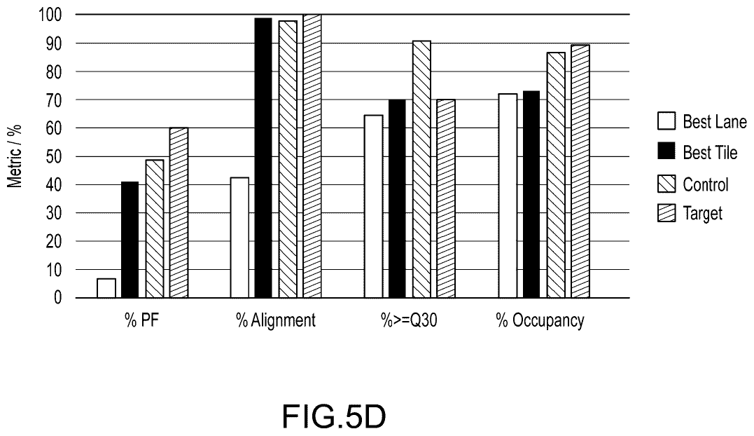

FIG. 5A illustrates Blackpool grafting of N₃-P5/P7 primers via “reverse” CuAAC chemistry to alkyne-PANA particles, resulting in PANA-P5P7 primer-grafted nanogel particles.

FIG. 5B illustrates capturing of PANA-P5P7 particles utilizing either of two different chemistries. The FC image was captured using the Typhoon at 580 nm via a CFR QC. Lanes 1 and 8 were control lanes, where standard primers were grafted to standard a PAZAM surface. Lanes 5-7 had PANA-P5P7 particles captured at different flush factors using CuAAC chemistry. Lanes 2-4 had PANA-P5P7 particles captured at different flush factors using DMTMM chemistry. For lanes 2-4, the grafting and capture was successful, as there was a significant intensity, 40-60%, compared to that of the control lanes. This was shown in previous studies to be sufficient to sustain a decent cluster growth). Unfortunately, for lanes 5-7, no significant intensity was detected, perhaps because the available —C≡CH groups were previously exhausted during the CuAAC reaction with N₃-P5/P7 primers. Nevertheless, this flow cell underwent standard on-board clustering and SBS on the HiSeqX™ platform.

In these working examples, particle capture of primer-grafter particles (PANA-P5P7) was only successfully at 60° C. in deionized water (pH 6.5). This is believed to be due to the particles shrinking the most at 60° C. in deionized water (to an average particle size d_{z}=175 nm). In comparison, particle size is about 240 nm at 30° C. Furthermore, the PANA particle sizes vary when measured in phosphate buffer (pH=7.4) from 220 nm to 250 nm. In deionized water (pH 6.5), the sizes changes from 175 nm to 225 nm. This indicated a pH responsiveness of the particles.

FIG. 5C shows the C1 intensity heat map of the FC compared to the CFR QC fluorescence intensity shown in FIG. 5B. In FIG. 5C, the C1 intensity heat map of the FC corroborates to the CFR QC fluorescence intensity observed and shown in FIG. 5B. According to lanes 2-4 where PANA-P5P7 particles are captured at different flush factors using DMTMM chemistry, the C1 intensity heat map shows that these lanes have been successfully sequenced. The target values for a successful SBS set of metrics, including % PF, % Alignment, %≥Q30 and % Occupancy were: 60, 100, 70 and 90%, respectively.

FIG. 5D sets forth SBS intensities and metrics obtained. Preliminary sequencing data demonstrate the compatibility of the material generated as a support for SBS. According to FIG. 5D, adequate % PF, % Alignment, %≥Q30 and % Occupancy metrics were observed for Lane 2 for the best tile, 40.9, 98.9, 70.2 and 70.5% respectively. Furthermore, with no optimization, the data is equivalent with baseline for several metrics.

### Additional Concepts

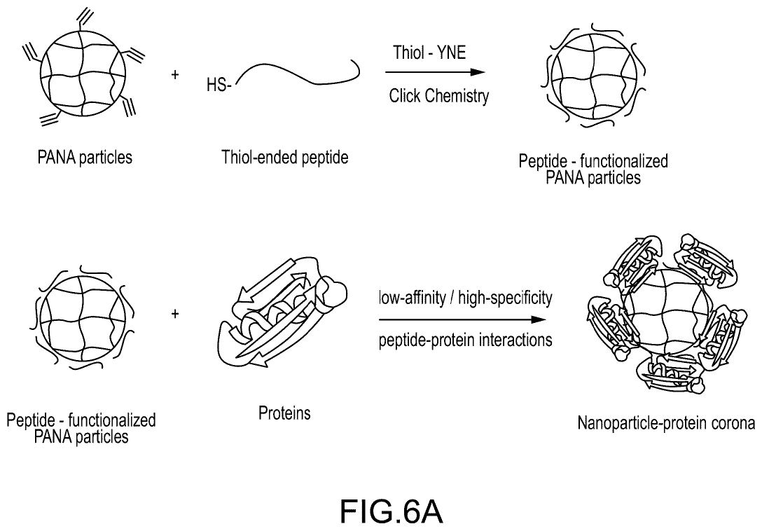

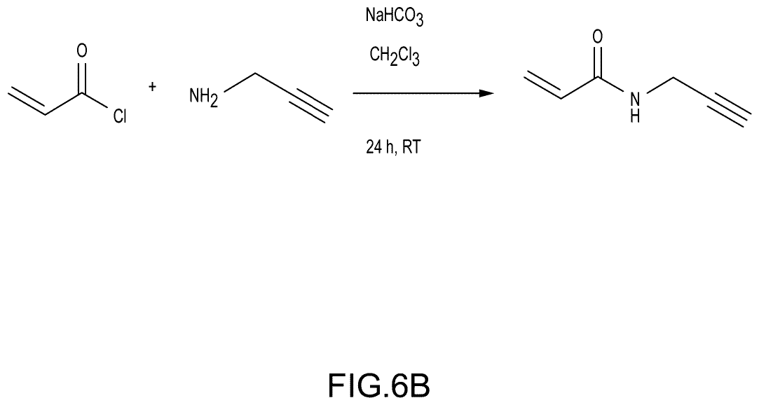

FIG. 6A details additional example concepts within the scope of the present disclosure.

As illustrated in FIG. 6A, thiol-alkyne reaction on PANA particles is a potential method for protein attachment and for future uses in proteomics using operations including:
(a) grafting thiol-terminated peptides onto PANA particles via thiol-alkyne click-chemistry;
(b) choosing peptides for low affinity/high specificity peptide-protein interactions; and
(c) forming various protein coronas to promote specific binding events.

FIG. 6B sets forth a synthesis of propargyl acrylamide (PAM), which is used to synthesize nanogel particles having —C≡CH functionality for use in the various concepts of FIG. 6A.

In the detailed description, references to “various examples”, “one example”, “an example”, etc., indicate that the example described may include a particular feature, structure, or characteristic, but every example may not necessarily include the particular feature, structure, or characteristic. Moreover, such phrases are not necessarily referring to the same example. Further, when a particular feature, structure, or characteristic is described in connection with an example, it is submitted that it is within the knowledge of one skilled in the art to affect such feature, structure, or characteristic in connection with other examples whether or not explicitly described. After reading the description, it will be apparent to one skilled in the relevant art(s) how to implement the disclosure in alternative examples.

Benefits, other advantages, and solutions to problems have been described herein with regard to specific examples. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to ‘at least one of A, B, and C’ or ‘at least one of A, B, or C’ is used in the claims or specification, it is intended that the phrase be interpreted to mean that A alone may be present in an example, B alone may be present in an example, C alone may be present in an example, or that any combination of the elements A, B and C may be present in a single example; for example, A and B, A and C, B and C, or A and B and C.

All structural, chemical, and functional equivalents to the elements of the above-described various examples that are known to those of ordinary skill in the art are expressly incorporated herein by reference and are intended to be encompassed by the present claims. Moreover, it is not necessary for an apparatus or component of an apparatus, or method in using an apparatus to address each and every problem sought to be solved by the present disclosure, for it to be encompassed by the present claims. Furthermore, no element, component, or method step in the present disclosure is intended to be dedicated to the public regardless of whether the element, component, or method step is explicitly recited in the claims. No claim element is intended to invoke 35 U.S.C. 112(f) unless the element is expressly recited using the phrase “means for.” As used herein, the terms “comprises”, “comprising”, or any other variation thereof, are intended to cover a non-exclusive inclusion, such that a chemical, chemical composition, process, method, article, or apparatus that comprises a list of elements does not include only those elements but may include other elements not expressly listed or inherent to such chemical, chemical composition, process, method, article, or apparatus.

### Additional Comments

While various illustrative examples are described above, it will be apparent to one skilled in the art that various changes and modifications may be made therein without departing from the invention. The appended claims are intended to cover all such changes and modifications that fall within the true spirit and scope of the invention.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

1. A polymeric nanogel particle comprising:
copolymer chains further comprising a first recurring unit of Formula (I), 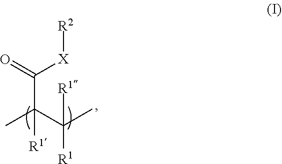
wherein each of R¹, R^{1′}, and R^{1″} is independently selected from H, halogen, alkyl, alkoxy, alkenyl, alkynyl, cycloalkyl, aryl, heteroaryl, or heterocyclyl; X is —O— or —NH—; and R² is —CH₂—C≡CH or has a structure: 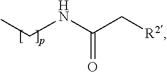
wherein R^{2′} is —N₃ or —C≡CH; and p is an integer of 1 to 50;
and a second recurring unit of Formula (II), 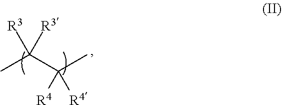
wherein each of R³, R^{3′}, R⁴, and R^{4′} is independently selected from —H, —R⁵, —OR⁵, —CO₂R⁵, —C(O)R⁵, —OC(O)R⁵, —C(O)NR⁶R⁷, and —NR⁶R⁷; wherein R⁵ is —H, —OH, alkyl, cycloalkyl, hydroxyalkyl, aryl, heteroaryl, or heterocyclyl; and each of R⁶ and R⁷ is independently selected from —H and alkyl; and
wherein at least some of the copolymer chains include at least one carboxylic acid end group; and wherein at least some of the copolymer chains include at least one —N₃ or —C≡CH end group.

2. The polymeric nanogel particle of claim 1, wherein R¹, R^{1′}, and R^{1″} are H.

3. The polymeric nanogel particle of claim 1, wherein R¹ and R^{1″} are H, and R^{1′} is CH₃.

4. The polymeric nanogel particle of claim 1, wherein the first recurring unit of Formula (I) is:

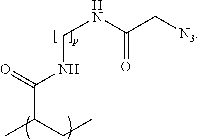

5. The polymeric nanogel particle of claim 1, wherein the first recurring unit of Formula (I) is:

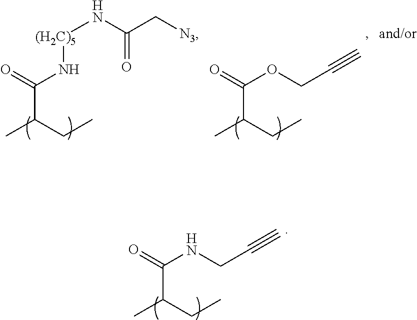

6. The polymeric nanogel particle of claim 1, wherein the second recurring unit of Formula (II) is at least one of:

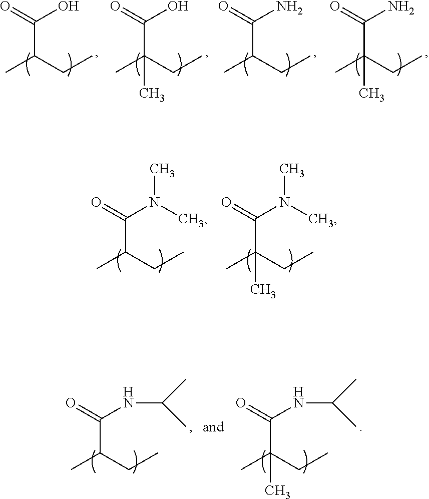

7. (canceled)

8. The polymeric nanogel particle of claim 1, wherein the copolymer chains comprise poly(AzAPA-co-NiPAM-co-AAc-co-BisAM) copolymer chains, wherein AzAPA is the monomer N-(5-(2-azidoacetamido)pentyl)acrylamide, NiPAM is the monomer N-isopropylacrylamide, AAc is the monomer acrylic acid, and BisAM is the monomer N,N′-methylenebisacrylamide.

9. (canceled)

10. The polymeric nanogel particle of claim 1, wherein the copolymer chains comprise poly(PAG-co-NiPAM-co-AAc-co-BisAM) and/or poly(PAM-co-NiPAM-co-AAc-co-BisAM) copolymer chains, wherein PAG is the monomer propargyl acrylate, PAM is the monomer N-propargyl acrylamide, NiPAM is the monomer N-isopropylacrylamide, AAc is the monomer acrylic acid, and BisAM is the monomer N,N′-methylenebisacrylamide.

11. A substrate having a surface comprising a plurality of pH and temperature responsive organic polymeric nanogel particles covalently attached thereto, wherein the organic polymeric nanogel particles include a plurality of copolymer chains comprising:
copolymer chains further comprising a first recurring unit of Formula (I), 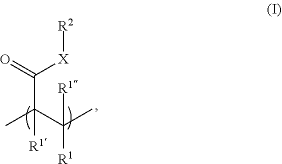
wherein each of R¹, R^{1′}, and R^{1″} is independently selected from H, halogen, alkyl, alkoxy, alkenyl, alkynyl, cycloalkyl, aryl, heteroaryl, or heterocyclyl; X is —O— or —NH—; and R² is —CH₂—C≡CH or has a structure: 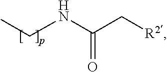
wherein R^{2′} is —N₃ or —C≡CH; and p is an integer of 1 to 50;
and a second recurring unit of Formula (II), 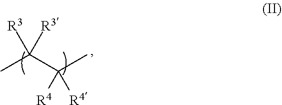
wherein each of R³, R^{3′}, R⁴, and R^{4′} is independently selected from —H, —R⁵, —OR⁵, —CO₂R⁵, —C(O)R⁵, —OC(O)R⁵, —C(O)NR⁶R⁷, and —NR⁶R⁷; wherein R⁵ is —H, —OH, alkyl, cycloalkyl, hydroxyalkyl, aryl, heteroaryl, or heterocyclyl; and each of R⁶ and R⁷ is independently selected from —H and alkyl; and
wherein at least some of the copolymer chains include at least one carboxylic acid end group; and wherein at least some of the copolymer chains include at least one —N₃ or —C≡CH end group.

12. The substrate of claim 11, wherein the copolymer chains comprise the repeating units (a) propargyl acrylate (PAG) and/or N-propargyl acrylamide (PAM), (b) N-isopropylacrylamide (NiPAM); and (c) acrylic acid (AAc).

13. The substrate of claim 12, wherein the organic polymer nanogel particles have an average size of about 265 nm to about 280 nm.

14. The substrate of claim 11, wherein the copolymer chains comprise the repeating units N-(5-(2-azidoacetamido)pentyl)acrylamide (AzAPA), N-isopropylacrylamide (NiPAM), and acrylic acid (AAc).

15. The substrate of claim 14, wherein the organic polymer nanogel particles have an average size of about 225 nm to about 250 nm.

16. The substrate of claim 11, wherein the copolymer chains comprise crosslinks between copolymer chains, and wherein said crosslinks comprise a multi-functional compound selected from the group consisting of N,N′-methylenebisacrylamide, N,N′-methylenebismethacrylamide, polyethylene glycol diacrylate, polyethylene glycol dimethacrylate, N-vinylacrylamide, glycidyl acrylate, divinylbenzene, tetraallyl ammonium chloride, diallyl dimethyl ammonium chloride, and mixtures thereof.

17. The substrate of claim 11, wherein the covalent attachment between the substrate and the plurality of nanogel particles comprise amide —NH—C(O)— linkages, wherein the —NH— portion of each amide linkage originated as an —NH₂ group present in a plurality of —NH₂ groups on the substrate, and wherein the —C(O)— portion of each amide linkage originated as a carboxylic acid end group on a respective copolymer chain.

18. The substrate of claim 11, wherein the organic polymeric nanogel particles further comprise amplification primers grafted thereon.

19. The substrate of claim 18, wherein each graft of an amplification primer to an organic polymeric nanogel particle comprises a triazine linkage, formed from a click-chemistry reaction between a terminal alkyne substituent on the amplification primer and an azide group on an end of a respective copolymer chain, or a click-chemistry reaction between a terminal azide substituent on the amplification primer and an alkyne group on an end of a respective copolymer chain.

20. The substrate of claim 11, wherein the first recurring unit of Formula (I) is at least one of:

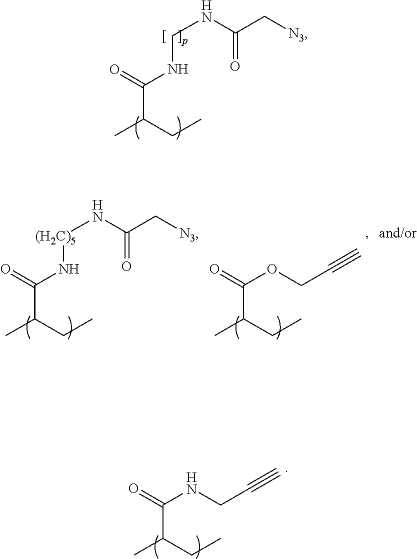

21. (canceled)

22. The substrate of claim 11, wherein the second recurring unit of Formula (II) is at least one of:

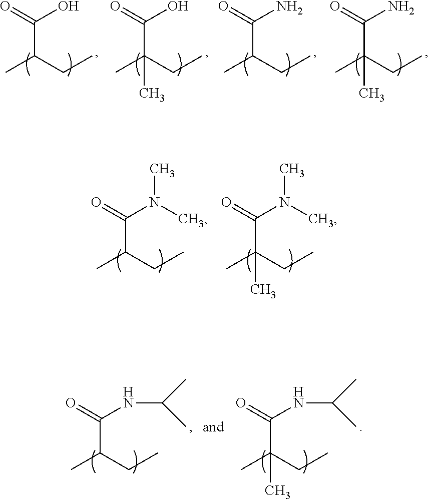

23. A flow cell comprising the substrate of claim 11, wherein the organic polymeric nanogel particles further comprise amplification primers grafted thereon.

24-35. (canceled)
